(19) United States
(12) Patent Application Publication
(10) Pub. No.: US 2022/0296367 A1
(43) Pub. Date: Sep. 22, 2022

(54) FRAME WITH VARIED STRUT WIDTHS FOR PROSTHETIC IMPLANT

(71) Applicant: Edwards Lifesciences Corporation, Irvine, CA (US)

(72) Inventors: Lien Huong Thi Hoang, San Juan Capistrano, CA (US); Venkateswaran Shanmugam, Lake Forest, CA (US)

(21) Appl. No.: 17/805,019

(22) Filed: Jun. 1, 2022

Related U.S. Application Data
(63) Continuation of application No. PCT/US2020/062644, filed on Dec. 1, 2020.
(60) Provisional application No. 62/942,704, filed on Dec. 2, 2019.

(51) Int. Cl. A61F 2/24

(52) U.S. Cl. CPC A61F 2/2418; A61F 2/2433; A61F 2250/001; A61F 2/9517

(57) ABSTRACT

A prosthetic implant has a self-expanding frame with an inflow end, an outflow end, and a plurality of struts interconnected at junctions. At least a portion of the plurality of struts have a reduced strut width at at least one junction configured to reduce or prevent infolding of the frame during recapture into a delivery cylinder of a delivery apparatus.

### CROSS REFERENCE TO RELATED APPLICATION

This application is a continuation of International Patent Application No. PCT/US2020/062644, filed on Dec. 1, 2020, which application claims the benefit of U.S. Provisional Application No. 62/942,704, filed on Dec. 2, 2019. The entire disclosure of each of these applications is incorporated herein by reference in its entirety.

### FIELD

The present disclosure pertains to prosthetic implants, such as self-expanding prosthetic heart valves and support structures, as well as associated delivery apparatuses.

### BACKGROUND

Prosthetic cardiac valves have been used for many years to treat cardiac valvular disorders. The native heart valves (such as the aortic, pulmonary and mitral valves) serve critical functions in assuring the forward flow of an adequate supply of blood through the cardiovascular system. These heart valves can be rendered less effective by congenital, inflammatory or infectious conditions. Such damage to the valves can result in serious cardiovascular compromise or death. For many years the definitive treatment for such disorders was the surgical repair or replacement of the valve during open heart surgery, but such surgeries are prone to many complications. More recently a transvascular technique has been developed for introducing and implanting a prosthetic heart valve using a flexible catheter in a manner that is less invasive than open heart surgery.

In this technique, a prosthetic valve is mounted in a crimped state on the end portion of a flexible catheter and advanced through a blood vessel of the patient until the prosthetic valve reaches the implantation site. The prosthetic valve at the catheter tip is then expanded to its functional size at the site of the defective native valve such as by inflating a balloon on which the prosthetic valve is mounted. Alternatively, the prosthetic valve can have a resilient, self-expanding stent or frame that expands the prosthetic valve to its functional size when it is advanced from a delivery sheath at the distal end of the catheter.

Balloon-expandable prosthetic valves typically are preferred for replacing calcified native valves because the catheter balloon can apply sufficient expanding force to anchor the frame of the prosthetic valve to the surrounding calcified tissue. On the other hand, self-expanding prosthetic valves sometimes are preferred for replacing a defective, non-stenotic (non-calcified) native valve, although they also can be used to replace stenotic valves.

During implantation of a self-expanding implant such as a prosthetic valve or valve support stent, the surgeon may partially advance the implant from the delivery cylinder or sheath in which it is contained in order to assess the positioning of the implant before fully deploying it. If positional adjustment is needed, the surgeon may partially or fully retract the prosthetic implant back into the delivery sheath, a process known as “recapturing” the prosthetic implant. During implant recapture, the distal end portion of the delivery sheath can urge or guide the prosthetic implant back into the compressed state as the prosthetic implant is withdrawn back into the delivery sheath. Partial deployment and implant recapture may be performed multiple times to achieve the desired positioning before the prosthetic implant is fully deployed. However, certain self-expanding prosthetic implants, such as relatively large diameter prosthetic heart valves and support stents, can be prone to infolding in which one or more struts bend, deform, or buckle radially inwardly during recapture. Such infolding can result in a fold, crease, or pocket in the exterior of the frame, necessitating replacement of the prosthetic implant and/or balloon valvuloplasty to fully expand the prosthetic implant after deployment. Accordingly, there exists a need for improvements to frames for self-expanding prosthetic implants such as prosthetic heart valves and support stents.

### SUMMARY

Certain embodiments of the disclosure pertain to self-expanding frames for prosthetic implants with varying strut widths, thicknesses, junction widths, and other parameters configured to reduce or prevent infolding of the frames during recapture into a delivery cylinder of a delivery apparatus. In a representative embodiment, a prosthetic implant comprises a self-expanding frame having an inflow end, an outflow end, and a plurality of struts, the struts being interconnected at junctions, and wherein at least a portion of the plurality of struts have a reduced strut width at at least one junction.

In any or all of the disclosed embodiments, the struts of the at least a portion of the plurality of struts have a reduced strut width at both junctions.

In any or all of the disclosed embodiments, the struts of the at least a portion of the plurality of struts have a reduced strut width at their inflow junctions.

In any or all of the disclosed embodiments, the struts of the at least a portion of the plurality of struts have a reduced strut width at their outflow junctions.

In any or all of the disclosed embodiments, struts of at least the second row of struts comprise a reduced strut width at their outflow junctions.

In any or all of the disclosed embodiments, the struts of at least the second row of struts comprise a reduced strut width at their inflow junctions.

In any or all of the disclosed embodiments, the struts define a first row of struts at the inflow end of the frame, a second row of struts at the outflow end of the frame, and at least one row of struts between the inflow end and the outflow end of the frame.

In any or all of the disclosed embodiments, struts of at least the first row of struts comprise a reduced strut width at their inflow junctions.

In any or all of the disclosed embodiments, struts of at least the first row of struts comprise a reduced strut width at their outflow junctions.

In any or all of the disclosed embodiments, the struts comprise inflow end portions, outflow end portions, and intermediate portions between the inflow end portions and the outflow end portions, wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width.

In any or all of the disclosed embodiments, the third strut width is greater than the first strut width and greater than the second strut width.

In any or all of the disclosed embodiments, the first strut width and the second strut width are substantially equal.

In any or all of the disclosed embodiments, a ratio of the first strut width to the third strut width is less than or equal to 0.95, or from 0.7 to 0.95.

In any or all of the disclosed embodiments, a ratio of the second strut width to the third strut width is less than or equal to 0.95, or from 0.7 to 0.95.

In any or all of the disclosed embodiments, a thickness of the struts is greater than the third strut width.

In any or all of the disclosed embodiments, a ratio of the third strut width to the strut thickness is greater than or equal to 0.65, or from 0.65 to 0.85.

In any or all of the disclosed embodiments, the junctions comprise a junction width, and the junction width is greater than the third strut width.

In any or all of the disclosed embodiments, a ratio of the third strut width to the junction width is 0.3 to 0.5.

In any or all of the disclosed embodiments, the struts comprise a strut thickness, and the junction width is greater than the strut thickness.

In any or all of the disclosed embodiments, a ratio of the junction width to the strut thickness is less than or equal to 2.1, or from 1.5 to 2.1.

In any or all of the disclosed embodiments, when 80% of an overall length of the prosthetic implant is deployed from a delivery cylinder of a delivery apparatus, a ratio of a diameter of the inflow end of the prosthetic implant to an inner diameter of the delivery cylinder is less than or equal to 6.0, or 5.0 to 6.0.

In any or all of the disclosed embodiments, the inflow end portions of the struts of the second row of struts comprise the first strut width, the outflow end portions of the struts of the second row of struts comprise the second strut width, and the intermediate portions of the struts of the second row of struts comprise the third strut width.

In any or all of the disclosed embodiments, each junction comprises a curved inflow surface, the curved inflow surface defining a radius, and a ratio of the second strut width of the outflow ends of the struts to the radius of the curved inflow surface is 4.0 to 7.5.

In any or all of the disclosed embodiments, all struts of the frame comprise the first strut width, the second strut width, and the third strut width.

In any or all of the disclosed embodiments, all struts of the frame comprise the first strut width, the second strut width, and the third strut width.

In any or all of the disclosed embodiments, the prosthetic implant is a prosthetic heart valve comprising a plurality of leaflets coupled to the frame and configured to regulate a flow of blood through the frame.

In any or all of the disclosed embodiments, the prosthetic implant is a docking station configured to be implanted in an annulus of a native heart valve, and configured to receive a prosthetic heart valve.

In another representative embodiment, a method comprises advancing the prosthetic implant of any embodiment described herein from a delivery cylinder of a delivery apparatus in which the prosthetic implant is retained in a radially compressed state such that the inflow end of the prosthetic implant at least partially expands, and retracting the prosthetic implant back into the delivery cylinder such that the prosthetic implant returns to the radially compressed state.

In another representative embodiment, a prosthetic implant delivery apparatus comprises a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant according to any of the embodiments described herein retained in a radially compressed state in the delivery cylinder.

In any or all of the disclosed embodiments, the prosthetic implant comprises a specified design diameter of at least 29 mm, and when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

In another representative embodiment, a prosthetic implant comprises a self-expanding frame having an inflow end, an outflow end, and a plurality of struts, the struts being interconnected at junctions, wherein the struts define a first row of struts at the inflow end of the frame, a second row of struts at the outflow end of the frame, and at least one row of struts between the inflow end and the outflow end of the frame. The struts comprise inflow end portions, outflow end portions, and intermediate portions between the inflow end portions and the outflow end portions. The inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width and greater than the second strut width.

In another representative embodiment, a prosthetic implant comprises a self-expanding frame having an inflow end, an outflow end, and a plurality of struts, the struts being interconnected at junctions. The struts comprise inflow end portions coupled to respective junctions, outflow end portions coupled to respective junctions, and intermediate portions between the inflow end portions and the outflow end portions. A strut width of the intermediate portions of the struts is different from a strut width of the inflow end portions of the struts and different from a strut width of the outflow end portions of the struts. The struts comprise a strut thickness. A ratio of the strut width of the intermediate portions of the struts to the strut thickness is greater than or equal to 0.65, or 0.65 to 0.85.

In another representative embodiment, a prosthetic implant comprises a self-expanding frame having an inflow end, an outflow end, and a plurality of struts, the struts being interconnected at junctions, the junctions comprising a junction width. The struts comprise inflow end portions coupled to respective junctions, outflow end portions coupled to respective junctions, and intermediate portions between the inflow end portions and the outflow end portions. The inflow end portions of the struts comprise a first strut width, the outflow end portions of the struts comprise a second strut width, and the intermediate portions of the struts comprise a third strut width that is greater than the first strut width and greater than the second strut width. The junction width is greater than the third strut width of the intermediate portions of the struts.

In another representative embodiment, a prosthetic implant delivery apparatus comprises a catheter comprising a handle portion at a proximal end portion of the catheter and an elongated shaft extending from the handle portion, the catheter further comprising a delivery cylinder at a distal end portion of the shaft, the delivery cylinder comprising an inner diameter. A self-expanding prosthetic implant retained in a radially compressed state in the delivery cylinder, the prosthetic implant comprising a self-expanding frame having an inflow end, an outflow end, and a plurality of struts, the struts being interconnected at junctions. The prosthetic implant has a specified design diameter of at least 29 mm. When the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a prosthetic valve that can be used to replace the native aortic valve of the heart, according to one embodiment.

FIG. 2 is a perspective view of a portion of the prosthetic valve of FIG. 1 illustrating the connection of two leaflets to the support frame of the prosthetic valve.

FIG. 3 is side elevation view of the support frame of the prosthetic valve of FIG. 1.

FIG. 4 is a perspective view of the support frame of the prosthetic valve of FIG. 1.

FIG. 5A is a cross-sectional view of the heart showing the prosthetic valve of FIG. 1 implanted within the aortic annulus.

FIG. 5B is an enlarged view of FIG. 5A illustrating the prosthetic valve implanted within the aortic annulus, shown with the leaflet structure of the prosthetic valve removed for clarity.

FIG. 6 is a perspective view of the leaflet structure of the prosthetic valve of FIG. 1 shown prior to being secured to the support frame.

FIG. 7 is a cross-sectional view of the prosthetic valve of FIG. 1.

FIG. 8 is a cross-sectional view of an embodiment of a delivery apparatus that can be used to deliver and implant a prosthetic valve, such as the prosthetic valve shown in FIG. 1. FIGS. 8A-8C are enlarged cross-sectional views of sections of FIG. 8.

FIG. 9 is an exploded view of the delivery apparatus of FIG. 8.

FIG. 10 is a side view of the guide catheter of the delivery apparatus of FIG. 8.

FIG. 11 is a perspective, exploded view of the proximal end portion of the guide catheter of FIG. 10.

FIG. 12 is a perspective, exploded view of the distal end portion of the guide catheter of FIG. 10.

FIG. 13 is a side view of the torque shaft catheter of the delivery apparatus of FIG. 8.

FIG. 14 is an enlarged side view of the rotatable screw of the torque shaft catheter of FIG. 13.

FIG. 15 is an enlarged perspective view of a coupling member disposed at the end of the torque shaft.

FIG. 16 is an enlarged perspective view of the threaded nut used in the torque shaft catheter of FIG. 13.

FIG. 17 is an enlarged side view of the distal end portion of the nose cone catheter of the delivery apparatus of FIG. 8.

FIG. 17A is an enlarged, cross-sectional view of the nose cone of the catheter shown FIG. 17.

FIG. 17B is an enlarged cross-sectional view of the distal end portion of the delivery apparatus of FIG. 8 showing the stent of a prosthetic valve retained in a compressed state within a delivery sheath.

FIG. 18 is an enlarged side view of the distal end portion of the delivery apparatus of FIG. 8 showing the delivery sheath in a delivery position covering a prosthetic valve in a compressed state for delivery into a patient.

FIG. 19 is an enlarged cross-sectional view of a section of the distal end portion of the delivery apparatus of FIG. 8 showing the valve-retaining mechanism securing the stent of a prosthetic valve to the delivery apparatus.

FIG. 20 is an enlarged cross-sectional view similar to FIG. 19, showing the inner fork of the valve-retaining mechanism in a release position for releasing the prosthetic valve from the delivery apparatus.

FIGS. 21 and 22 are enlarged side views of distal end portion of the delivery apparatus of FIG. 8, illustrating the operation of the torque shaft for deploying a prosthetic valve from a delivery sheath.

FIGS. 23-26 are various views of an embodiment of a motorized delivery apparatus that can be used to operate the torque shaft of the delivery apparatus shown in FIG. 8.

FIG. 27 is a perspective view of an alternative motor that can be used to operate the torque shaft of the delivery apparatus shown in FIG. 8.

FIG. 28A is an enlarged view of a distal segment of the guide catheter shaft of FIG. 10.

FIG. 28B shows the cut pattern for forming the portion of the shaft shown in FIG. 28A, such as by laser cutting a metal tube.

FIG. 29A is an enlarged view of a distal segment of a guide catheter shaft, according to another embodiment.

FIG. 29B shows the cut pattern for forming the shaft of FIG. 29A, such as by laser cutting a metal tube.

FIG. 30 is a side elevation view of a support stent for use in a prosthetic valve.

FIG. 31 is a side elevation view of a frame of a prosthetic heart valve partially deployed from a delivery cylinder.

FIGS. 32-35 are perspective views of a distal end portion of a prosthetic heart valve partially deployed from a delivery cylinder and inverting as the prosthetic heart valve is retracted into the delivery cylinder.

FIG. 36 is a side elevation view of a frame for a prosthetic heart valve, according to another embodiment.

FIG. 37 is a magnified view of a portion of a strut row of the frame of FIG. 36.

FIG. 38 is a side elevation view of a junction between two strut rows of the frame of FIG. 36.

FIG. 39 is a side elevation view of the frame of 36 partially deployed from a delivery cylinder.

FIG. 40 is a side elevation view of the frame of FIG. 36 illustrating the overall length Y of the frame at the specified design diameter.

FIG. 41 is a graph showing radial force as a function of diameter for the frame of FIG. 36.

FIGS. 42-44 are top perspective views illustrating recapture of the frame of FIG. 36 without infolding.

FIG. 45 is a perspective view of a radial expansion force gauge apparatus, according to one embodiment.

FIG. 46 is a rear end view of the apparatus of FIG. 45 with a calibration yoke and weights attached.

FIGS. 47A-51 illustrate an embodiment of a self-expanding docking station configured to receive a prosthetic heart valve, according to one embodiment.

FIGS. 52-53B illustrate another embodiment of a self-expanding docking station configured to receive a prosthetic heart valve.

FIGS. 54 and 55 illustrate another embodiment of a self-expanding prosthetic heart valve.

FIG. 56 illustrates another embodiment of a self-expanding prosthetic heart valve.

FIG. 57 illustrates a portion of a junction between two strut rows of the frame of FIG. 36, according to another embodiment.

FIGS. 58 and 59 illustrate a portion of a junction between two strut rows of the frame of FIG. 36, according to another embodiment.

FIG. 60 is a side elevation view of a frame of a self-expanding docking station configured to receive a prosthetic heart valve, according to another embodiment.

FIG. 61 is a perspective view of another embodiment of a prosthetic heart valve configured for implantation in the native mitral valve.

FIG. 62 is a perspective view of an inner frame of the prosthetic heart valve of FIG. 61, according to one embodiment.

FIG. 63 is a is a perspective view of an outer frame of the prosthetic heart valve of FIG. 61, according to one embodiment.

FIG. 64 illustrates the outer frame of the prosthetic heart valve of FIG. 61 in a laid-flat configuration.

### DETAILED DESCRIPTION

Described herein are embodiments of self-expanding frames for prosthetic implants with varying strut widths, thicknesses, junction widths, and/or other parameters configured to reduce or prevent infolding of the frames during recapture into a delivery cylinder/sheath of a delivery apparatus. For example, in certain embodiments, struts of the frames described herein can comprise strut widths at or near junctions between adjacent struts that are less than strut widths near the centers of the struts. In certain embodiments, a ratio of the strut width at or near the junctions to the strut width at the mid-portion of the struts within particular ranges can reduce the incidence of infolding during recapture of the frame. In certain embodiments, the struts can have a reduced strut width at their inflow junctions, at their outflow junctions, or both. In certain embodiments, the struts of the row or rows of struts at the inflow end of the frame can comprise varying strut widths as described herein. In certain embodiments, varying the strut widths as described herein can maintain a ratio of the inflow diameter of a partially deployed frame to the inner diameter of a delivery cylinder within a specified range in order to reduce infolding. For example, certain frame embodiments described herein can allow 80% or more of the overall length of the frame to be unsheathed from a delivery cylinder and then recaptured into the delivery cylinder without infolding. In certain examples, this can reduce the risk that the implant may become damaged and require replacement mid-procedure, thereby reducing procedure time and improving patient outcomes.

### First Representative Embodiment

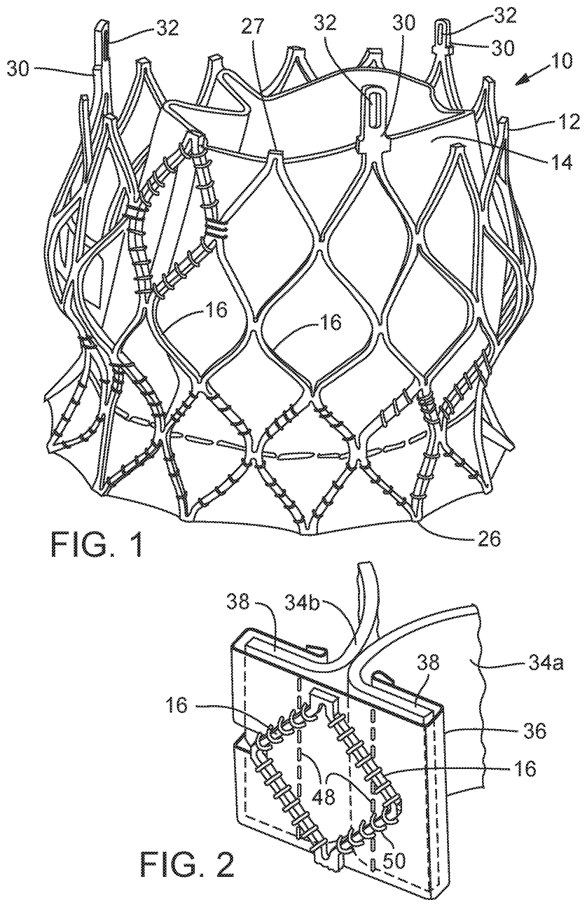

Referring first to FIG. 1, there is shown a prosthetic aortic heart valve 10, according to one embodiment. The prosthetic valve 10 includes an expandable frame member, or stent, 12 that supports a flexible leaflet section 14. The prosthetic valve 10 is radially compressible to a compressed state for delivery through the body to a deployment site and expandable to its functional size shown in FIG. 1 at the deployment site. In certain embodiments, the prosthetic valve 10 is self-expanding; that is, the prosthetic valve can radially expand to its functional size when advanced from the distal end of a delivery sheath. Apparatuses particularly suited for percutaneous delivery and implantation of a self-expanding prosthetic valve are described in detail below. In other embodiments, the prosthetic valve can be a balloon-expandable prosthetic valve that can be adapted to be mounted in a compressed state on the balloon of a delivery catheter. The prosthetic valve can be expanded to its functional size at a deployment site by inflating the balloon, as known in the art.

The illustrated prosthetic valve 10 is adapted to be deployed in the native aortic annulus, although it also can be used to replace the other native valves of the heart. Moreover, the prosthetic valve 10 can be adapted to replace other valves within the body, such venous valves.

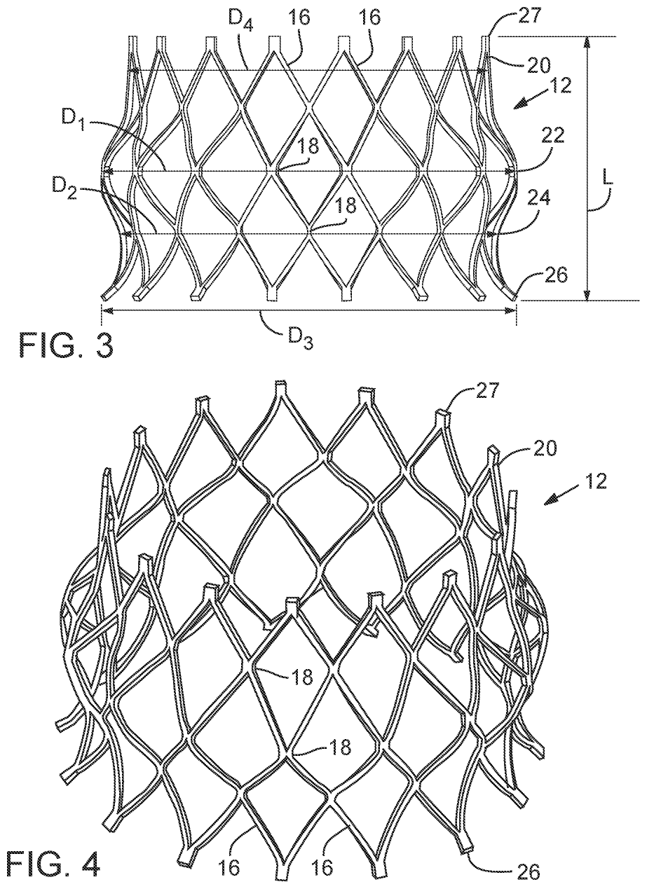

FIGS. 3 and 4 show the stent 12 without the leaflet section 14 for purposes of illustration. As shown, the stent 12 can be formed from a plurality of longitudinally extending, generally sinusoidal shaped frame members, or struts, 16. The struts 16 are formed with alternating bends and are welded or otherwise secured to each other at nodes 18 formed from the vertices of adjacent bends so as to form a mesh structure. The struts 16 can be made of a suitable shape memory material, such as the nickel titanium alloy known as Nitinol, that allows the prosthetic valve to be compressed to a reduced diameter for delivery in a delivery apparatus (such as described below) and then causes the prosthetic valve to expand to its functional size inside the patient's body when deployed from the delivery apparatus. If the prosthetic valve is a balloon-expandable prosthetic valve that is adapted to be crimped onto an inflatable balloon of a delivery apparatus and expanded to its functional size by inflation of the balloon, the stent 12 can be made of a suitable ductile material, such as nickel-chromium alloys or stainless steel.

The stent 12 has an inflow end 26 and an outflow end 27. The mesh structure formed by struts 16 comprises a generally cylindrical “upper” or outflow end portion 20, an outwardly bowed or distended intermediate section 22, and an inwardly bowed “lower” or inflow end portion 24. The intermediate section 22 desirably is sized and shaped to extend into the Valsalva sinuses in the root of the aorta to assist in anchoring the prosthetic valve in place once implanted. As shown, the mesh structure desirably has a curved shape along its entire length that gradually increases in diameter from the outflow end portion 20 to the intermediate section 22, then gradually decreases in diameter from the intermediate section 22 to a location on the inflow end portion 24, and then gradually increases in diameter to form a flared portion terminating at the inflow end 26.

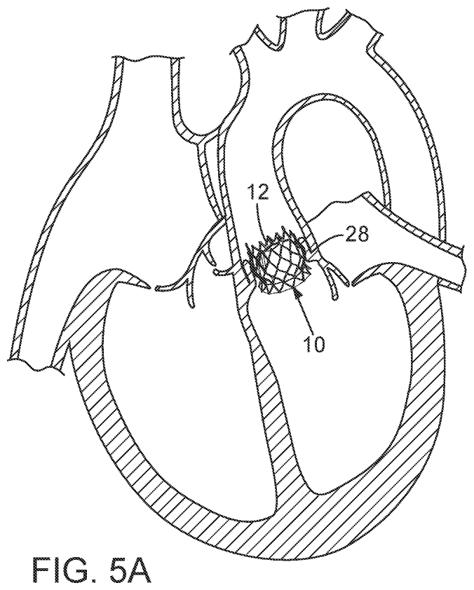

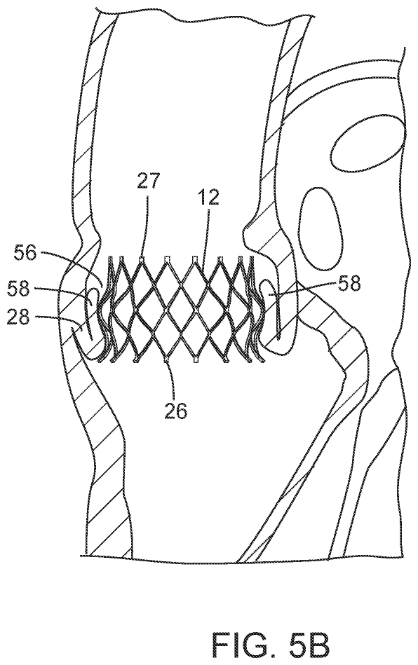

When the prosthetic valve is in its expanded state, the intermediate section 22 has a diameter D₁, the inflow end portion 24 has a minimum diameter D₂, the inflow end 26 has a diameter D₃, and the outflow end portion 20 has a diameter D₄, where D₂ is less than D₁ and D₃, and D₄ is less than D₂. In addition, D₁ and D₃ desirably are greater than the diameter of the native annulus in which the prosthetic valve is to be implanted. In this manner, the overall shape of the stent 12 assists in retaining the prosthetic valve at the implantation site. More specifically, and referring to FIGS. 5A and 5B, the prosthetic valve 10 can be implanted within a native valve (the aortic valve in the illustrated example) such that the lower section 24 is positioned within the aortic annulus 28, the intermediate section 24 extends above the aortic annulus into the Valsalva's sinuses 56, and the lower flared end 26 extends below the aortic annulus. The prosthetic valve 10 is retained within the native valve by the radial outward force of the lower section 24 against the surrounding tissue of the aortic annulus 28 as well as the geometry of the stent. Specifically, the intermediate section 24 and the flared lower end 26 extend radially outwardly beyond the aortic annulus 28 to better resist against axial dislodgement of the prosthetic valve in the upstream and downstream directions (toward and away from the aorta). Depending on the condition of the native leaflets 58, the prosthetic valve typically is deployed within the native annulus 28 with the native leaflets 58 folded upwardly and compressed between the outer surface of the stent 12 and the walls of the Valsalva sinuses, as depicted in FIG. 5B. In some cases, it may be desirable to excise the leaflets 58 prior to implanting the prosthetic valve 10.

Known prosthetic valves having a self-expanding frame typically have additional anchoring devices or frame portions that extend into and become fixed to non-diseased areas of the vasculature. Because the shape of the stent 12 assists in retaining the prosthetic valve, additional anchoring devices are not required and the overall length L of the stent can be minimized to prevent the stent upper portion 20 from extending into the non-diseased area of the aorta, or to at least minimize the extent to which the upper portion 20 extends into the non-diseased area of the aorta. Avoiding the non-diseased area of the patient's vasculature helps avoid complications if future intervention is required. For example, the prosthetic valve can be more easily removed from the patient because the stent is primarily anchored to the diseased part of the native valve. Furthermore, in certain embodiments a shorter prosthetic valve can be more easily navigated around the aortic arch.

In particular embodiments, for a prosthetic valve intended for use in a 22-mm to 24-mm annulus, the diameter D1 is about 28 mm to about 32 mm, with 30 mm being a specific example; the diameter D2 is about 24 mm to about 28 mm, with 26 mm being a specific example; the diameter D3 is about 28 mm to about 32 mm, with 30 mm being a specific example; and the diameter D4 is about 24 mm to about 28 mm, with 26 mm being a specific example. The length L in particular embodiments is about 20 mm to about 24 mm, with 22 mm being a specific example.

Referring to FIG. 1, the stent 12 can have a plurality of angularly spaced retaining arms, or projections, in the form of posts 30 (three in the illustrated embodiment) that extend from the stent upper portion 20. Each retaining arm 30 has a respective aperture 32 that is sized to receive prongs of a valve-retaining mechanism that can be used to form a releasable connection between the prosthetic valve and a delivery apparatus (described below). In alternative embodiments, the retaining arms 30 need not be provided if a valve-retaining mechanism is not used.

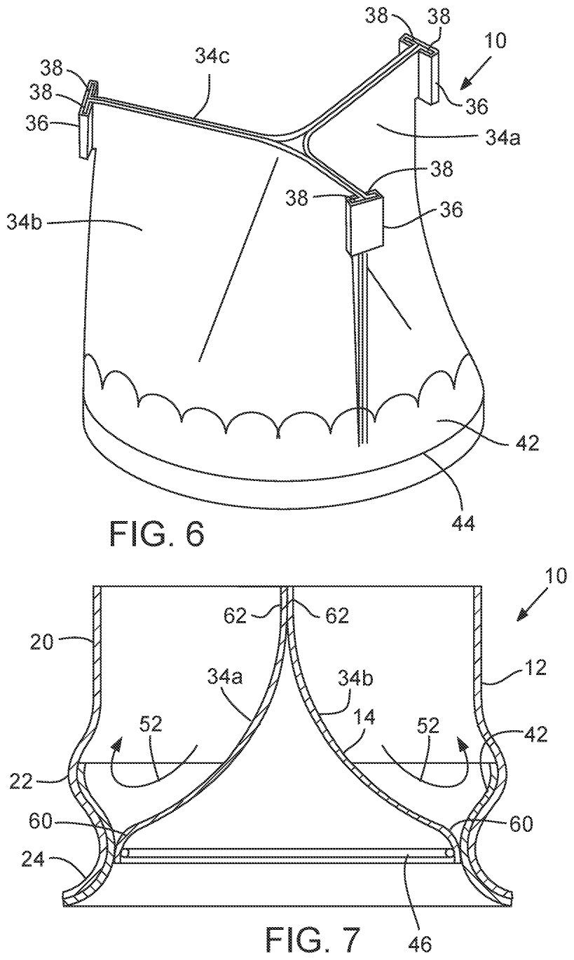

As best shown in FIGS. 6 and 7, the leaflet assembly 14 in the illustrated embodiment comprises three leaflets 34a, 34b, 34c made of a flexible material. Each leaflet has an inflow end portion 60 and an outflow end portion 62. The leaflets can comprise any suitable biological material (e.g., pericardial tissue, such as bovine or equine pericardium), bio-compatible synthetic materials, or other such materials, such as those described in U.S. Pat. No. 6,730,118, which is incorporated herein by reference. The leaflet assembly 14 can include an annular reinforcing skirt 42 that is secured to the outer surfaces of the inflow end portions of the leaflets 34a, 34b, 34c at a suture line 44 adjacent the inflow end of the prosthetic valve. The inflow end portion of the leaflet assembly 14 can be secured to the stent 12 by suturing the skirt 42 to struts 16 of the lower section 24 of the stent (best shown in FIG. 1). As shown in FIG. 7, the leaflet assembly 14 can further include an inner reinforcing strip 46 that is secured to the inner surfaces of the inflow end portions 60 of the leaflets.

Referring to FIGS. 1 and 2, the outflow end portion of the leaflet assembly 14 can be secured to the upper portion of the stent 12 at three angularly spaced commissure attachments of the leaflets 34a, 34b, 34c. As best shown in FIG. 2, each commissure attachment can be formed by wrapping a reinforcing section 36 around adjacent upper edge portions 38 of a pair of leaflets at the commissure formed by the two leaflets and securing the reinforcing section 36 to the edge portions 38 with sutures 48. The sandwiched layers of the reinforcing material and leaflets can then be secured to the struts 16 of the stent 12 with sutures 50 adjacent the outflow end of the stent. The leaflets therefore desirably extend the entire length or substantially the entire length of the stent from the inflow end 26 to the outflow end 27. The reinforcing sections 36 reinforces the attachment of the leaflets to the stent so as to minimize stress concentrations at the suture lines and avoid “needle holes” on the portions of the leaflets that flex during use. The reinforcing sections 36, the skirt 42, and the inner reinforcing strip 46 desirably are made of a bio-compatible synthetic material, such as polytetrafluoroethylene (PTFE), or a woven fabric material, such as woven polyester (e.g., polyethylene terephtalate) (PET)).

FIG. 7 shows the operation of the prosthetic valve 10. During diastole, the leaflets 34a, 34b, 34c collapse to effectively close the prosthetic valve. As shown, the curved shape of the intermediate section 22 of the stent 12 defines a space between the intermediate section and the leaflets that mimics the Valsalva sinuses. Thus, when the leaflets close, backflow entering the “sinuses” creates a turbulent flow of blood along the upper surfaces of the leaflets, as indicated by arrows 52. This turbulence assists in washing the leaflets and the skirt 42 to minimize clot formation.

The prosthetic valve 10 can be implanted in a retrograde approach where the prosthetic valve, mounted in a crimped state at the distal end of a delivery apparatus, is introduced into the body via the femoral artery and advanced through the aortic arch to the heart, as further described in U.S. Patent Publication No. 2008/0065011, which is incorporated herein by reference.

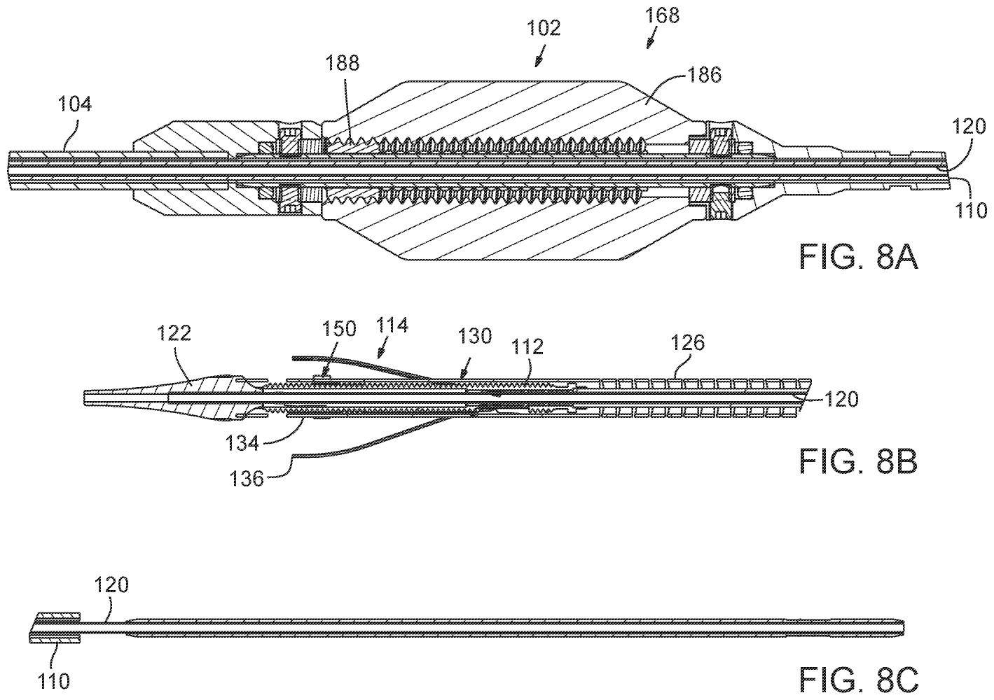

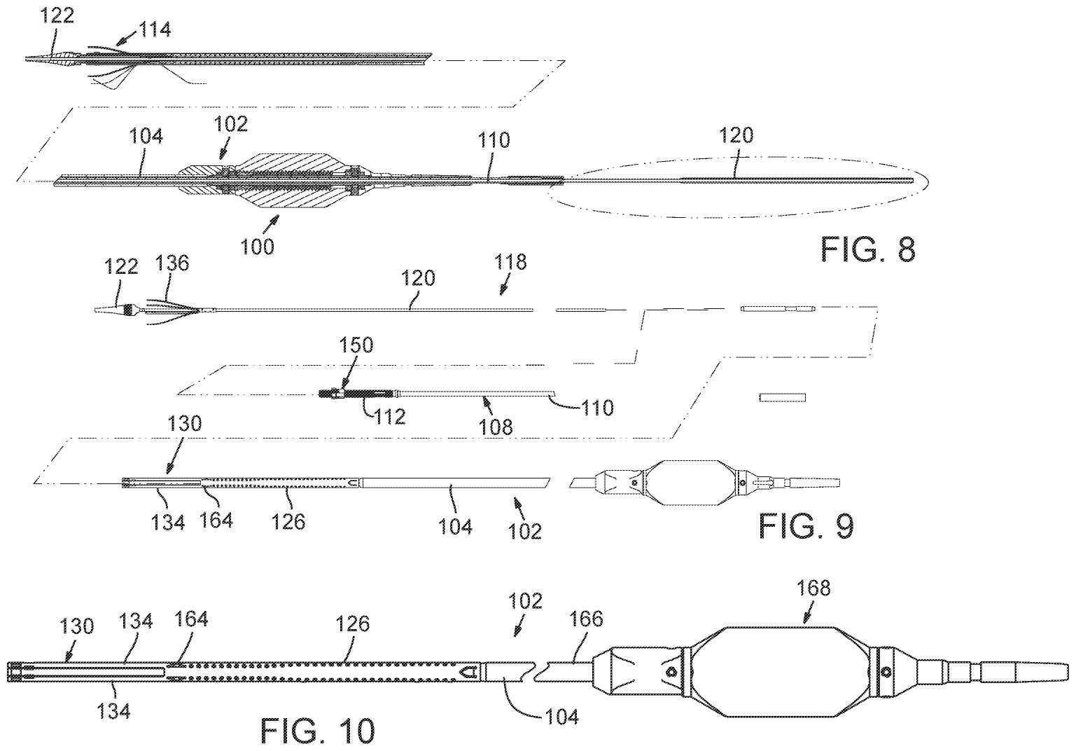

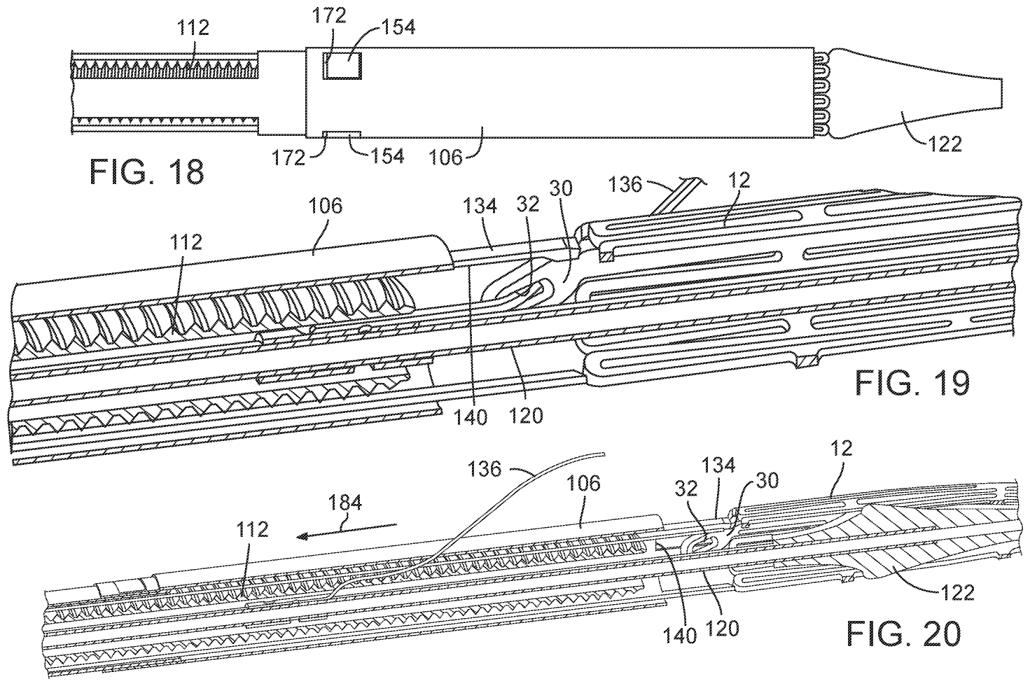

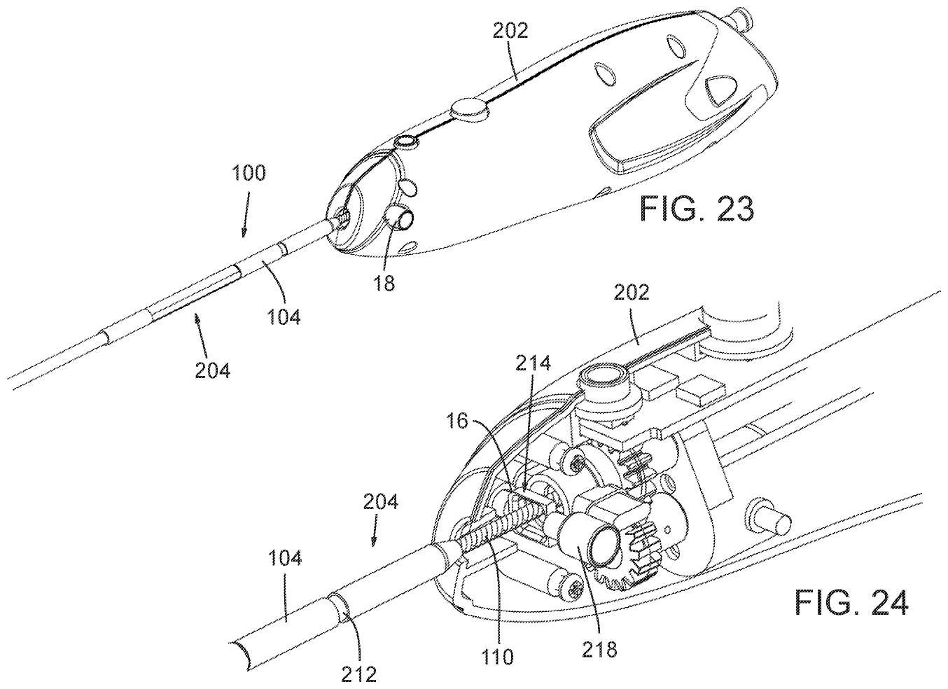

FIGS. 8 and 9 show a delivery apparatus 100, according to one embodiment, that can be used to deliver a self-expanding prosthetic valve, such as prosthetic valve 10 described above, through a patient's vasculature. The delivery apparatus 100 comprises a first, outermost or main catheter 102 (shown alone in FIG. 10) having an elongated shaft 104, the distal end of which is coupled to a delivery sheath 106 (FIG. 18; also referred to as a delivery cylinder). The proximal end of the main catheter 102 is connected to a handle of the delivery apparatus. FIGS. 23-26 show an embodiment of a handle mechanism having an electric motor for operating the delivery apparatus. The handle mechanism is described in detail below. During delivery of a prosthetic valve, the handle can be used by a surgeon to advance and retract the delivery apparatus through the patient's vasculature. Although not required, the main catheter 102 can comprise a guide catheter that is configured to allow a surgeon to guide or control the amount the bending or flexing of a distal portion of the shaft 104 as it is advanced through the patient's vasculature, such as further described below. Another embodiment of a guide catheter is disclosed in U.S. Patent Publication No. 2008/0065011, which is incorporated herein by reference.

As best shown in FIG. 9, the delivery apparatus 100 also includes a second, intermediate catheter 108 (also referred to herein as a torque shaft catheter) having an elongated shaft 110 (also referred to herein as a torque shaft) and an elongated screw 112 connected to the distal end of the shaft 110. The shaft 110 of the intermediate catheter 108 extends coaxially through the shaft 104 of the main catheter 102. The delivery apparatus 100 can also include a third, nose-cone catheter 118 having an elongated shaft 120 and a nose piece, or nose cone, 122 secured to the distal end portion of the shaft 120. The nose piece 122 can have a tapered outer surface as shown for atraumatic tracking through the patient's vasculature. The shaft 120 of the nose-cone catheter extends through the prosthetic valve 10 (not shown in FIGS. 8-9) and the shaft 110 of the intermediate catheter 108. In the illustrated configuration, the innermost shaft 120 is configured to be moveable axially and rotatably relative to the shafts 104, 110, and the torque shaft 110 is configured to be rotatable relative to the shafts 104, 120 to effect valve deployment and release of the prosthetic valve from the delivery apparatus, as described in detail below. Additionally, the innermost shaft 120 can have a lumen for receiving a guide wire so that the delivery apparatus can be advanced over the guide wire inside the patient's vasculature (FIG. 8C).

As best shown in FIG. 10, the outer catheter 102 can comprise a flex control mechanism 168 at a proximal end thereof to control the amount the bending or flexing of a distal portion of the outer shaft 104 as it is advanced through the patient's vasculature, such as further described below. The outer shaft 104 can comprise a proximal segment 166 that extends from the flex control mechanism 168 and a distal segment 126 that comprises a slotted metal tube that increases the flexibility of the outer shaft at this location. The distal end portion of the distal segment 126 can comprises an outer fork 130 of a valve-retaining mechanism 114 that is configured to releasably secure a prosthetic valve 10 to the delivery apparatus 100 during valve delivery, as described in detail below.

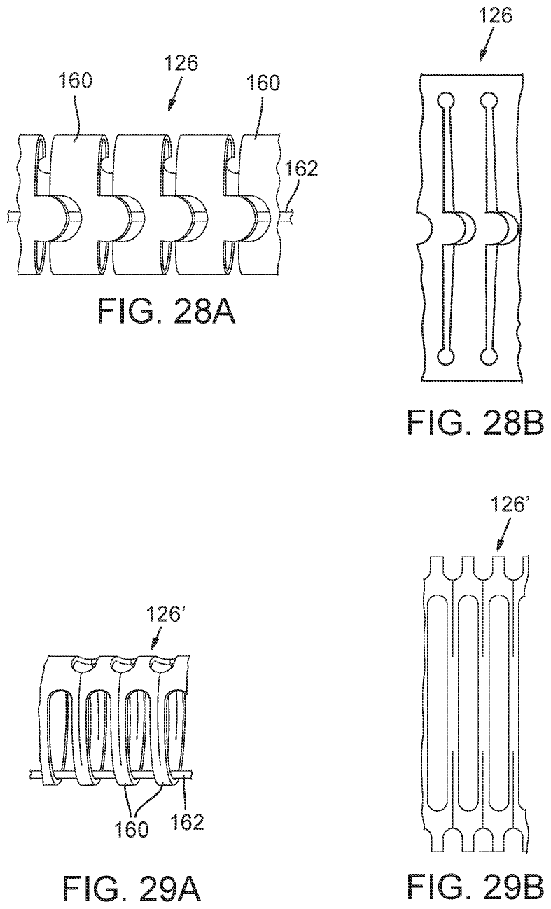

FIG. 28A is an enlarged view of a portion of the distal segment 126 of the outer shaft 104. FIG. 28B shows the cut pattern that can be used to form the distal segment 126 by laser cutting the pattern in a metal tube. The distal segment 126 comprises a plurality of interconnected circular bands or links 160 forming a slotted metal tube. A pull wire 162 can be positioned inside the distal segment 126 and can extend from a location 164 of the distal segment 126 (FIGS. 10 and 12) to the flex control mechanism. The distal end of the pull wire 162 can be secured to the inner surface of the distal segment 126 at location 164, such as by welding. The proximal end of the pull wire 162 can be operatively connected to the flex control mechanism 168, which is configured to apply and release tension to the pull wire in order to control bending of the shaft, as further described below. The links 160 of the shaft and the gaps between adjacent links are shaped to allow bending of the shaft upon application of light pulling force on the pull wire 162. In the illustrated embodiment, as best shown in FIG. 12, the distal segment 126 is secured to a proximal segment 166 having a different construction (e.g., one or more layers of polymeric tubing). In the illustrated embodiment, the proximal segment 166 extends from the flex control mechanism 168 to the distal segment 126 and therefore makes up the majority of the length of the outer shaft 104. In alternative embodiments, the entire length or substantially the entire length of the outer shaft 104 can be formed from a slotted metal tube comprising one or more sections of interconnected links 160. In any case, the use of a main shaft having such a construction can allow the delivery apparatus to be highly steerable, especially when use in combination with a torque shaft having the construction shown in FIGS. 40 and 41 (described below).

The width of the links 160 can be varied to vary the flexibility of the distal segment along its length. For example, the links within the distal end portion of the slotted tube can be relatively narrower to increase the flexibility of the shaft at that location while the links within the proximal end portion of the slotted tube can be relatively wider so that the shaft is relatively less flexible at that location.

FIG. 29A shows an alternative embodiment of a distal segment, indicated at 126′, which can be formed, for example, by laser cutting a metal tube. The segment 126′ can comprise the distal segment of an outer shaft of a delivery apparatus (as shown in FIG. 12) or substantially the entire length of an outer shaft can have the construction shown in FIG. 29A. FIG. 29B shows the cut pattern for forming the segment 126′. In another embodiment, a delivery apparatus can include a composite outer shaft comprising a laser-cut metal tube laminated with a polymeric outer layer that is fused within the gaps in the metal layer. In one example, a composite shaft can comprise a laser cut metal tube having the cut pattern of FIGS. 29A and 29B and a polymeric outer layer fused in the gaps between the links 160 of the metal tube. In another example, a composite shaft can comprise a laser cut metal tube having the cut pattern of FIGS. 28A and 28B and a polymeric outer layer fused in the gaps between the links 160 of the metal tube. A composite shaft also can include a polymeric inner layer fused in the gaps between the links 160 of the metal tube.

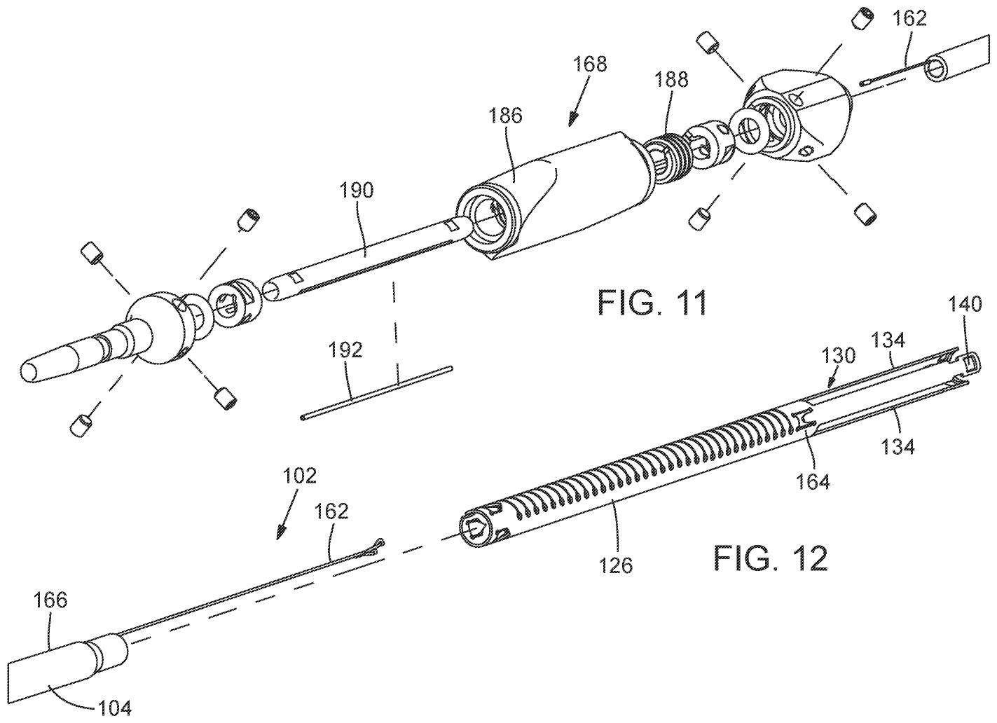

Referring to FIGS. 8A and 11, the flex control mechanism 168 can comprise a rotatable housing, or handle portion, 186 that houses a slide nut 188 mounted on a rail 192/190. The slide nut 188 is prevented from rotating within the housing by one or more rods 192, each of which is partially disposed in a corresponding recess within the rail 192 and a slot or recess on the inside of the nut 188. The proximal end of the pull wire 162 is secured to the nut 188. The nut 188 has external threads that engage internal threads of the housing. Thus, rotating the housing 186 causes the nut 188 to move axially within the housing in the proximal or distal direction, depending on the direction of rotation of the housing. Rotating the housing in a first direction (e.g., clockwise), causes the nut to travel in the proximal direction, which applies tension to the pull wire 162, which causes the distal end of the delivery apparatus to bend or flex. Rotating the housing in a second direction (e.g., counterclockwise), causes the nut to travel in the distal direction, which relieves tension in the pull wire 162 and allows the distal end of the delivery apparatus to flex back to its pre-flexed configuration under its own resiliency.

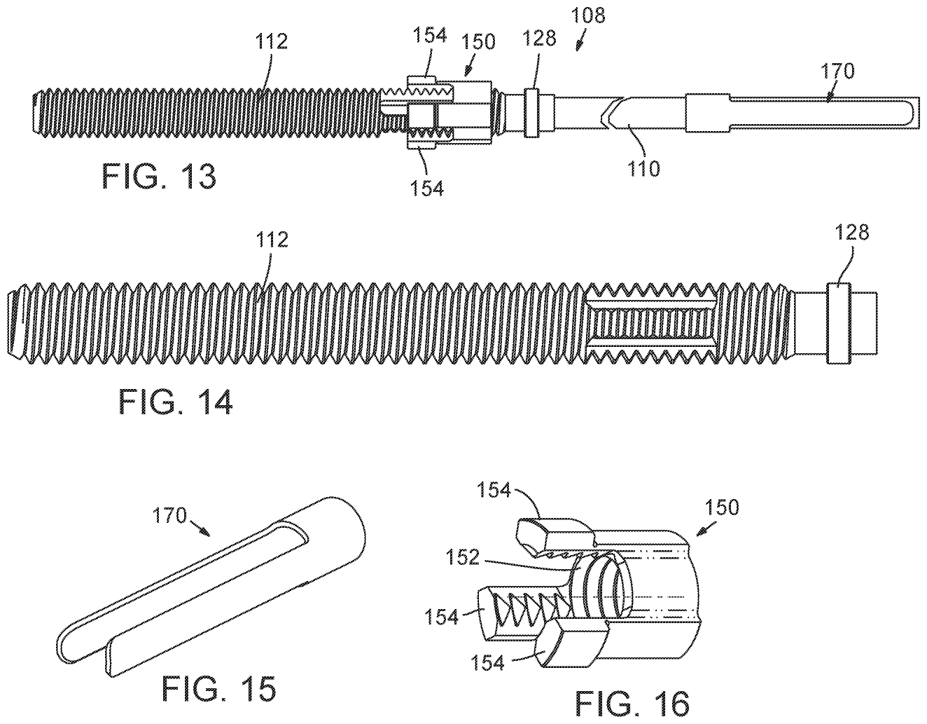

As best shown in FIG. 13, the torque shaft catheter 108 includes an annular projection in the form of a ring 128 (also referred to as an anchoring disc) mounted on the distal end portion of the torque shaft 110 adjacent the screw 112. The ring 128 is secured to the outer surface of the torque shaft 110 such that it cannot move axially or rotationally relative to the torque shaft. The inner surface of the outer shaft 104 is formed with a feature, such as a slot or recess, that receives the ring 128 in such a manner that the ring and the corresponding feature on the inner surface of the outer shaft 104 allow the torque shaft 110 to rotate relative to the outer shaft 104 but prevent the torque shaft from moving axially relative to the outer shaft. The corresponding feature on the outer shaft 104 that receives the ring 128 can be inwardly extending tab portions formed in the distal segment 126, such as shown at 164 in FIG. 12. In the illustrated embodiment (as best shown in FIG. 14), the ring 128 is an integral part of the screw 112 (i.e., the screw 112 and the ring 128 are portions of single component). Alternatively, the screw 112 and the ring are separately formed components but are both fixedly secured to the distal end of the torque shaft 110.

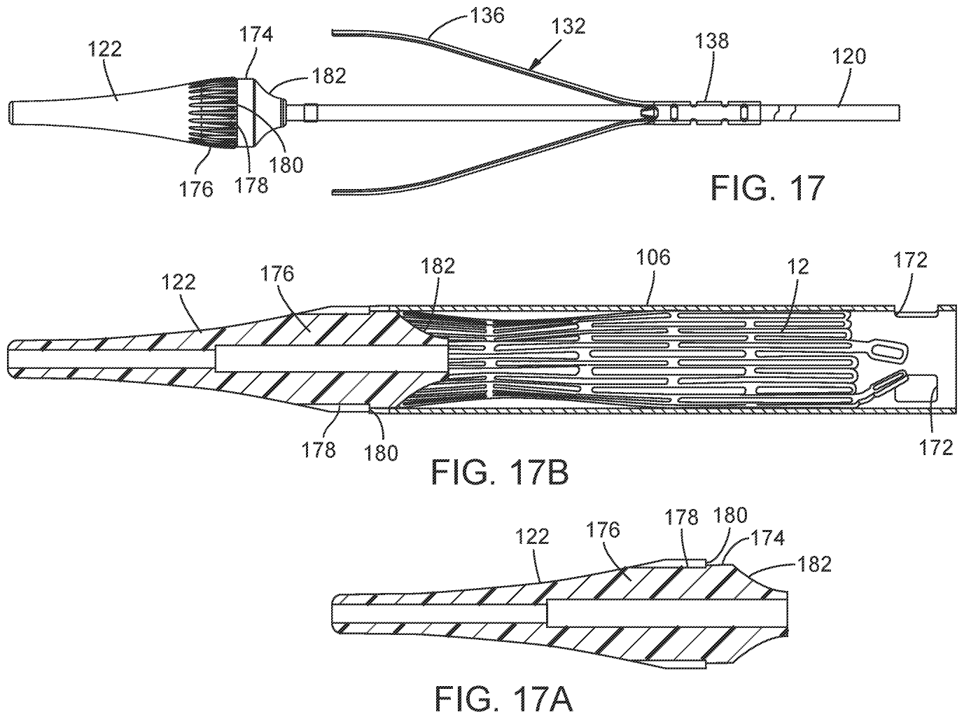

The torque shaft 110 desirably is configured to be rotatable relative to the delivery sheath 106 to effect incremental and controlled advancement of the prosthetic valve 10 from the delivery sheath 106. To such ends, and according to one embodiment, the delivery apparatus 100 can include a sheath retaining ring in the form of a threaded nut 150 mounted on the external threads of the screw 112. As best shown in FIG. 16, the nut 150 includes internal threads 152 that engage the external threads of the screw and axially extending legs 154. Each leg 154 has a raised distal end portion that extends into and/or forms a snap fit connection with openings 172 in the proximal end of the sheath 106 (as best shown in FIG. 18) so as to secure the sheath 106 to the nut 150. As illustrated in FIGS. 17B and 18, the sheath 106 extends over the prosthetic valve 10 and retains the prosthetic valve in a radially compressed state until the sheath 106 is retracted by the user to deploy the prosthetic valve.

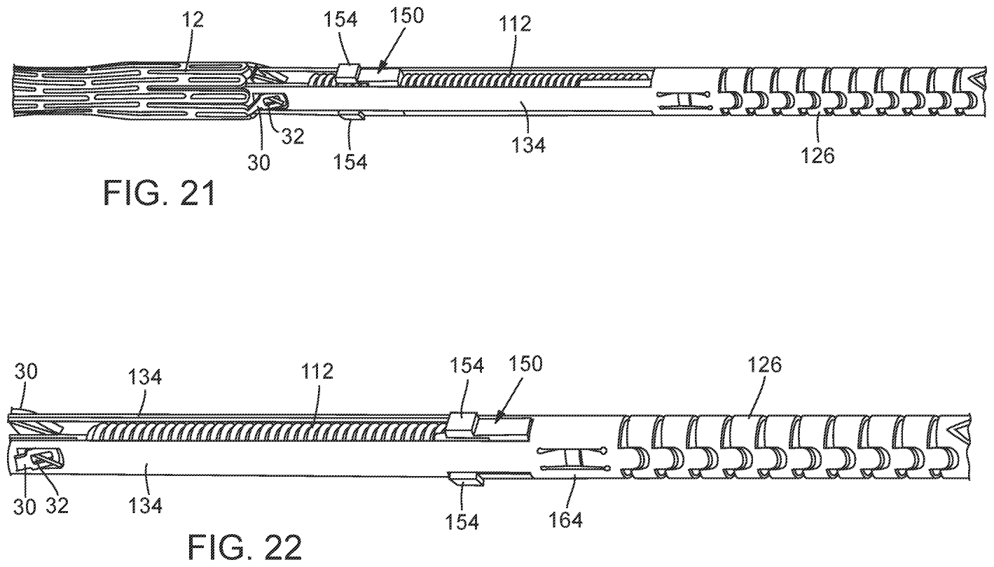

As best shown in FIGS. 21 and 22, the outer fork 130 of the valve-retaining mechanism comprises a plurality of prongs 134, each of which extends through a region defined between two adjacent legs 154 of the nut so as to prevent rotation of the nut relative to the screw 112 upon rotation of the screw. As such, rotation of the torque shaft 110 (and thus the screw 112) causes corresponding axial movement of the nut 150. The connection between the nut 150 and the sheath 106 is configured such that axially movement of the nut along the screw 112 (in the distal or proximal direction) causes the sheath 106 to move axially in the same direction relative to the screw and the valve-retaining mechanism. FIG. 21 shows the nut 150 in a distal position wherein the sheath 106 (not shown in FIG. 21) extends over and retains the prosthetic valve 10 in a compressed state for delivery. Movement of the nut 150 from the distal position (FIG. 21) to a proximal position (FIG. 22) causes the sheath 106 to move in the proximal direction, thereby deploying the prosthetic valve from the sheath 106. Rotation of the torque shaft 110 to effect axial movement of the sheath 106 can be accomplished with a motorized mechanism (such as shown in FIGS. 23-26 and described below) or by manually turning a crank or wheel.

FIG. 17 shows an enlarged view of the nose cone 122 secured to the distal end of the innermost shaft 120. The nose cone 122 in the illustrated embodiment includes a proximal end portion 174 that is sized to fit inside the distal end of the sheath 106. An intermediate section 176 of the nose cone is positioned immediately adjacent the end of the sheath in use and is formed with a plurality of longitudinal grooves, or recessed portions, 178. The diameter of the intermediate section 176 at its proximal end 180 desirably is slightly larger than the outer diameter of the sheath 106. The proximal end 180 can be held in close contact with the distal end of the sheath 106 to protect surrounding tissue from coming into contact with the metal edge of the sheath. The grooves 178 allow the intermediate section to be compressed radially as the delivery apparatus is advanced through an introducer sheath. This allows the nose cone to be slightly oversized relative to the inner diameter of the introducer sheath. FIG. 17B shows a cross-section the nose cone 122 and the sheath 106 in a delivery position with the prosthetic valve retained in a compressed delivery state inside the sheath 106 (for purposes of illustration, only the stent 12 of the prosthetic valve is shown). As shown, the proximal end 180 of the intermediate section 176 can abut the distal end of the sheath 106 and a tapered proximal surface 182 of the nose cone can extend within a distal portion of the stent 12.

As noted above, the delivery apparatus 100 can include a valve-retaining mechanism 114 (FIG. 8B) for releasably retaining a stent 12 of a prosthetic valve. The valve-retaining mechanism 114 can include a first valve-securement component in the form of an outer fork 130 (as best shown in FIG. 12) (also referred to as an “outer trident” or “release trident”), and a second valve-securement component in the form of an inner fork 132 (as best shown in FIG. 17) (also referred to as an “inner trident” or “locking trident”). The outer fork 130 cooperates with the inner fork 132 to form a releasable connection with the retaining arms 30 of the stent 12.

The proximal end of the outer fork 130 is connected to the distal segment 126 of the outer shaft 104 and the distal end of the outer fork is releasably connected to the stent 12. In the illustrated embodiment, the outer fork 130 and the distal segment 126 can be integrally formed as a single component (e.g., the outer fork and the distal segment can be laser cut or otherwise machined from a single piece of metal tubing), although these components can be separately formed and subsequently connected to each other. The inner fork 132 can be mounted on the nose catheter shaft 120 (as best shown in FIG. 17). The inner fork 132 connects the stent to the distal end portion of the nose catheter shaft 120. The nose catheter shaft 120 can be moved axially relative to the outer shaft 104 to release the prosthetic valve from the valve-retaining mechanism, as further described below.

As best shown in FIG. 12, the outer fork 130 includes a plurality of angularly-spaced prongs 134 (three in the illustrated embodiment) corresponding to the retaining arms 30 of the stent 12, which prongs extend from the distal end of distal segment 126. The distal end portion of each prong 134 includes a respective opening 140. As best shown in FIG. 17, the inner fork 132 includes a plurality of angularly-spaced prongs 136 (three in the illustrated embodiment) corresponding to the retaining arms 30 of the stent 12, which prongs extend from a base portion 138 at the proximal end of the inner fork. The base portion 138 of the inner fork is fixedly secured to the nose catheter shaft 120 (e.g., with a suitable adhesive) to prevent axial and rotational movement of the inner fork relative to the nose catheter shaft 120.

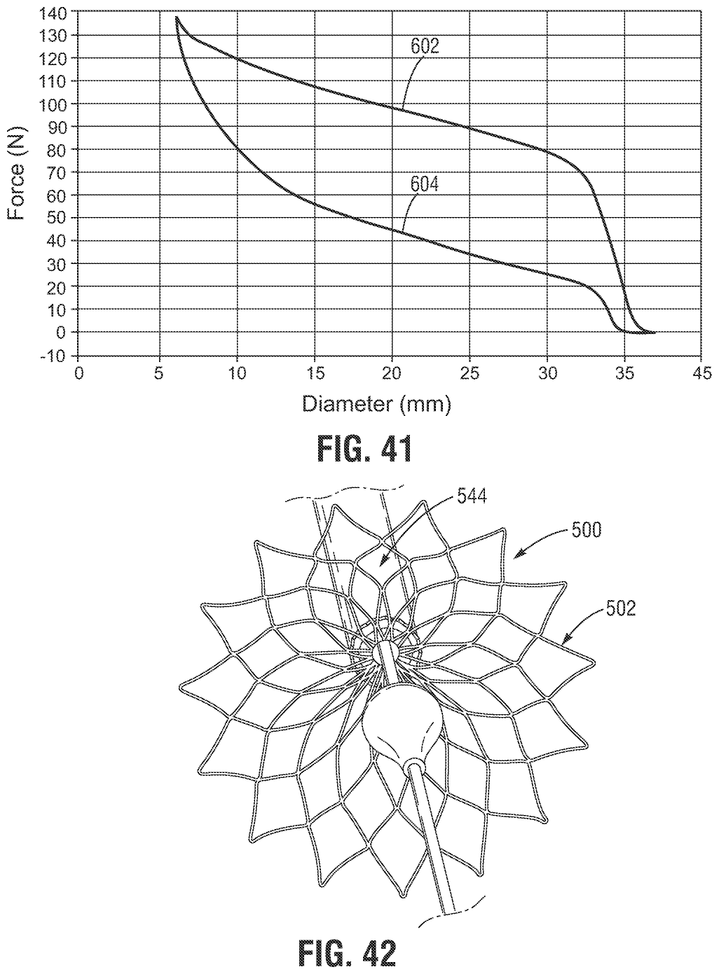

Each prong of the outer fork cooperates with a corresponding prong of the inner fork to form a releasable connection with a retaining arm 30 of the stent. In the illustrated embodiment, for example, the distal end portion of each prong 134 is formed with an opening 140. When the prosthetic valve is secured to the delivery apparatus (as best shown in FIG. 19), each retaining arm 30 of the stent 12 extends inwardly through an opening 140 of a prong 134 of the outer fork and a prong 136 of the inner fork is inserted through the opening 32 of the retaining arm 30 so as to retain the retaining arm 30 from backing out of the opening 140. FIG. 42 also shows the prosthetic valve 10 secured to the delivery apparatus by the inner and outer forks before the prosthetic valve is loaded into the sheath 106. Retracting the inner prongs 136 proximally (in the direction of arrow 184 in FIG. 20) to remove the prongs from the openings 32 is effective to release the prosthetic valve 10 from the retaining mechanism. When the inner fork 132 is moved to a proximal position (FIG. 20), the retaining arms 30 of the stent can move radially outwardly from the openings 140 in the outer fork 130 under the resiliency of the stent. In this manner, the valve-retaining mechanism 114 forms a releasable connection with the prosthetic valve that is secure enough to retain the prosthetic valve relative to the delivery apparatus to allow the user to fine tune or adjust the position of the prosthetic valve after it is deployed from the delivery sheath. When the prosthetic valve is positioned at the desired implantation site, the connection between the prosthetic valve and the retaining mechanism can be released by retracting the nose catheter shaft 120 relative to the outer shaft 104 (which retracts the inner fork 132 relative to the outer fork 130).

Techniques for compressing and loading the prosthetic valve 10 into the sheath 106 are described below. Once the prosthetic valve 10 is loaded in the delivery sheath 106, the delivery apparatus 100 can be inserted into the patient's body for delivery of the prosthetic valve. In one approach, the prosthetic valve can be delivered in a retrograde procedure where delivery apparatus is inserted into a femoral artery and advanced through the patient's vasculature to the heart. Prior to insertion of the delivery apparatus, an introducer sheath can be inserted into the femoral artery followed by a guide wire, which is advanced through the patient's vasculature through the aorta and into the left ventricle. The delivery apparatus 100 can then be inserted through the introducer sheath and advanced over the guide wire until the distal end portion of the delivery apparatus containing the prosthetic valve 10 is advanced to a location adjacent to or within the native aortic valve.

Thereafter, the prosthetic valve 10 can be deployed from the delivery apparatus 100 by rotating the torque shaft 110 relative to the outer shaft 104. As described below, the proximal end of the torque shaft 110 can be operatively connected to a manually rotatable handle portion or a motorized mechanism that allows the surgeon to effect rotation of the torque shaft 110 relative to the outer shaft 104. Rotation of the torque shaft 110 and the screw 112 causes the nut 150 and the sheath 106 to move in the proximal direction toward the outer shaft (FIG. 22), which deploys the prosthetic valve from the sheath. Rotation of the torque shaft 110 causes the sheath to move relative to the prosthetic valve in a precise and controlled manner as the prosthetic valve advances from the open distal end of the delivery sheath and begins to expand. Hence, unlike known delivery apparatuses, as the prosthetic valve begins to advance from the delivery sheath and expand, the prosthetic valve is held against uncontrolled movement from the sheath caused by the expansion force of the prosthetic valve against the distal end of the sheath. In addition, as the sheath 106 is retracted, the prosthetic valve 10 is retained in a stationary position relative to the ends of the inner shaft 120 and the outer shaft 104 by virtue of the valve-retaining mechanism 114. As such, the prosthetic valve 10 can be held stationary relative to the target location in the body as the sheath is retracted. Moreover, after the prosthetic valve is partially advanced from the sheath, it may be desirable to retract the prosthetic valve back into the sheath, for example, to reposition the prosthetic valve or to withdraw the prosthetic valve entirely from the body. The partially deployed prosthetic valve can be retracted back into the sheath by reversing the rotation of the torque shaft, which causes the sheath 106 to advance back over the prosthetic valve in the distal direction.

In known delivery devices, the surgeon must apply push-pull forces to the shaft and/or the sheath to unsheathe the prosthetic valve. It is therefore difficult to transmit forces to the distal end of the device without distorting the shaft (e.g., compressing or stretching the shaft axially), which in turn causes uncontrolled movement of the prosthetic valve during the unsheathing process. To mitigate this effect, the shaft and/or sheath can be made more rigid, which is undesirable because the device becomes harder to steer through the vasculature. In contrast, the manner of unsheathing the prosthetic valve described above eliminates the application of push-pull forces on the shaft, as required in known devices, so that relatively high and accurate forces can be applied to the distal end of the shaft without compromising the flexibility of the device. In certain embodiments, as much as 20 lbs. of force can be transmitted to the end of the torque shaft without adversely affecting the unsheathing process. In contrast, prior art devices utilizing push-pull mechanisms typically cannot exceed about 5 lbs. of force during the unsheathing process.

After the prosthetic valve 10 is advanced from the delivery sheath and expands to its functional size (the expanded prosthetic valve 10 secured to the delivery apparatus is depicted in FIG. 42 of U.S. Pat. No. 9,867,700, which is incorporated herein by reference), the prosthetic valve remains connected to the delivery apparatus via the retaining mechanism 114. Consequently, after the prosthetic valve is advanced from the delivery sheath, the surgeon can reposition the prosthetic valve relative to the desired implantation position in the native valve such as by moving the delivery apparatus in the proximal and distal directions or side to side, or rotating the delivery apparatus, which causes corresponding movement of the prosthetic valve. The retaining mechanism 114 desirably provides a connection between the prosthetic valve and the delivery apparatus that is secure and rigid enough to retain the position of the prosthetic valve relative to the delivery apparatus against the flow of the blood as the position of the prosthetic valve is adjusted relative to the desired implantation position in the native valve. Once the surgeon positions the prosthetic valve at the desired implantation position in the native valve, the connection between the prosthetic valve and the delivery apparatus can be released by retracting the innermost shaft 120 in the proximal direction relative to the outer shaft 104, which is effective to retract the inner fork 132 to withdraw its prongs 136 from the openings 32 in the retaining arms 30 of the prosthetic valve (FIG. 20). Slightly retracting of the outer shaft 104 allows the outer fork 130 to back off the retaining arms 30 of the prosthetic valve, which slide outwardly through openings 140 in the outer fork to completely disconnect the prosthetic valve from the retaining mechanism 114. Thereafter, the delivery apparatus can be withdrawn from the body, leaving the prosthetic aortic valve 10 implanted within the native valve (such as shown in FIGS. 5A and 5B).

The delivery apparatus 100 has at its distal end a semi-rigid segment comprised of relatively rigid components used to transform rotation of the torque shaft into axial movement of the sheath. In particular, this semi-rigid segment in the illustrated embodiment is comprised of the prosthetic valve and the screw 112. An advantage of the delivery apparatus 100 is that the overall length of the semi-rigid segment is minimized because the nut 150 is used rather than internal threads on the outer shaft to affect translation of the sheath. The reduced length of the semi-rigid segment increases the overall flexibility along the distal end portion of the delivery catheter. Moreover, the length and location of the semi-rigid segment remains constant because the torque shaft does not translate axially relative to the outer shaft. As such, the curved shape of the delivery catheter can be maintained during valve deployment, which improves the stability of the deployment. A further benefit of the delivery apparatus 100 is that the ring 128 prevents the transfer of axial loads (compression and tension) to the section of the torque shaft 110 that is distal to the ring.

In an alternative embodiment, the delivery apparatus can be adapted to deliver a balloon-expandable prosthetic valve. As described above, the valve retaining mechanism 114 can be used to secure the prosthetic valve to the end of the delivery apparatus. Since the stent of the prosthetic valve is not self-expanding, the sheath 106 can be optional. The retaining mechanism 114 enhances the pushability of the delivery apparatus and prosthetic valve assembly through an introducer sheath.

FIGS. 23-26 illustrate the proximal end portion of the delivery apparatus 100, according to one embodiment. The delivery apparatus 100 can comprise a handle 202 that is configured to be releasably connectable to the proximal end portion of a catheter assembly 204 comprising catheters 102, 108, 118. It may be desirable to disconnect the handle 202 from the catheter assembly 204 for various reasons. For example, disconnecting the handle can allow another device to be slid over the catheter assembly, such as a valve-retrieval device or a device to assist in steering the catheter assembly. It should be noted that any of the features of the handle 202 and the catheter assembly 204 can be implemented in any of the embodiments of the delivery apparatuses disclosed herein.

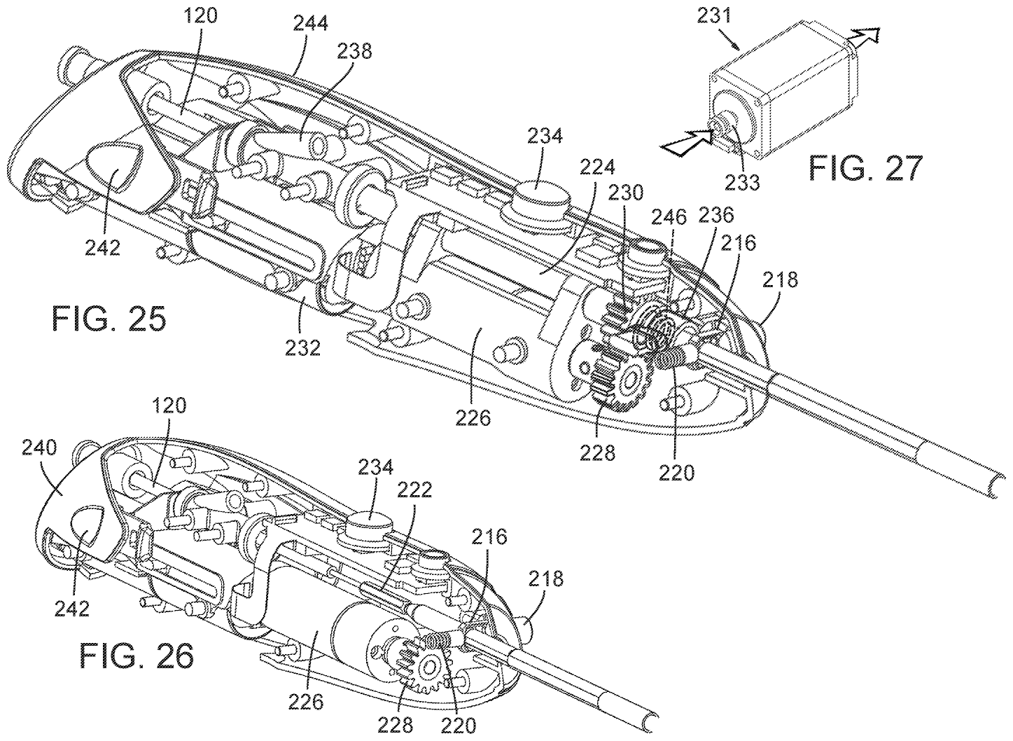

FIGS. 23 and 24 show the proximal end portion of the catheter assembly 204 partially inserted into a distal opening of the handle 202. The proximal end portion of the main shaft 104 is formed with an annular groove 212 (as best shown in FIG. 24) that cooperates with a holding mechanism, or latch mechanism, 214 inside the handle. When the proximal end portion of the catheter assembly is fully inserted into the handle, as shown in FIGS. 25 and 26, an engaging portion 216 of the holding mechanism 214 extends at least partially into the groove 212. One side of the holding mechanism 214 is connected to a button 218 that extends through the housing of the handle. The opposite side of the holding mechanism 214 is contacted by a spring 220 that biases the holding mechanism to a position engaging the main shaft 104 at the groove 212. The engagement of the holding mechanism 214 within the groove 212 prevents axial separation of the catheter assembly from the handle. The catheter assembly can be released from the handle by depressing button 218, which moves the holding mechanism 214 from locking engagement with the main shaft. Furthermore, the main shaft 104 can be formed with a flat surface portion within the groove 212. The flat surface portion is positioned against a corresponding flat surface portion of the engaging portion 216. This engagement holds the main shaft 104 stationary relative to the torque shaft 110 as the torque shaft is rotated during valve deployment.

The proximal end portion of the torque shaft 110 can have a driven nut 222 (FIG. 26) that is slidably received in a drive cylinder 224 (FIG. 25) mounted inside the handle. The nut 222 can be secured to the proximal end of the torque shaft 100 by securing the nut 222 over a coupling member 170 (FIG. 15). FIG. 26 is a perspective view of the inside of the handle 202 with the drive cylinder and other components removed to show the driven nut and other components positioned within the drive cylinder. The cylinder 224 has a through opening (or lumen) extending the length of the cylinder that is shaped to correspond to the flats of the nut 222 such that rotation of the drive cylinder is effective to rotate the nut 222 and the torque shaft 110. The drive cylinder can have an enlarged distal end portion 236 that can house one or more seals (e.g., o-rings 246) that form a seal with the outer surface of the main shaft 104 (FIG. 25). The handle can also house a fitting 238 that has a flush port in communication with the lumen of the torque shaft and/or the lumen of the main shaft.

The drive cylinder 224 is operatively connected to an electric motor 226 through gears 228 and 230. The handle can also house a battery compartment 232 that contains batteries for powering the motor 226. Rotation of the motor in one direction causes the torque shaft 110 to rotate, which in turn causes the sheath 106 to retract and uncover a prosthetic valve at the distal end of the catheter assembly. Rotation of the motor in the opposite direction causes the torque shaft to rotate in an opposite direction, which causes the sheath to move back over the prosthetic valve. An operator button 234 on the handle allows a user to activate the motor, which can be rotated in either direction to un-sheath a prosthetic valve or retrieve an expanded or partially expanded prosthetic valve.

As described above, the distal end portion of the nose catheter shaft 120 can be secured to an inner fork 132 that is moved relative to an outer fork 130 to release a prosthetic valve secured to the end of the delivery apparatus. Movement of the shaft 120 relative to the main shaft 104 (which secures the outer fork 130) can be effected by a proximal end portion 240 of the handle that is slidable relative to the main housing 244. The end portion 240 is operatively connected to the shaft 120 such that movement of the end portion 240 is effective to translate the shaft 120 axially relative to the main shaft 104 (causing a prosthetic valve to be released from the inner and outer forks). The end portion 240 can have flexible side panels 242 on opposite sides of the handle that are normally biased outwardly in a locked position to retain the end portion relative to the main housing 244. During deployment of the prosthetic valve, the user can depress the side panels 242, which disengage from corresponding features in the housing and allow the end portion 240 to be pulled proximally relative to the main housing, which causes corresponding axial movement of the shaft 120 relative to the main shaft. Proximal movement of the shaft 120 causes the prongs 136 of the inner fork 132 to disengage from the apertures 32 in the stent 12, which in turn allows the retaining arms 30 of the stent to deflect radially outwardly from the openings 140 in the prongs 134 of the outer fork 130, thereby releasing the prosthetic valve.

FIG. 27 shows an alternative embodiment of a motor, indicated at 231, that can be used to drive a torque shaft (e.g., torque shaft 110). In this embodiment, a catheter assembly can be connected directly to one end of a shaft 233 of the motor, without gearing. The shaft 233 includes a lumen that allows for passage of an innermost shaft (e.g., shaft 120) of the catheter assembly, a guide wire, and/or fluids for flushing the lumens of the catheter assembly.

Alternatively, the power source for rotating the torque shaft 110 can be a hydraulic power source (e.g., hydraulic pump) or pneumatic (air-operated) power source that is configured to rotate the torque shaft. In another embodiment, the handle can have a manually movable lever or wheel that is operable to rotate the torque shaft 110.

In another embodiment, a power source (e.g., an electric, hydraulic, or pneumatic power source) can be operatively connected to a shaft, which is turn is connected to a prosthetic valve 10. The power source is configured to reciprocate the shaft longitudinally in the distal direction relative to a valve sheath in a precise and controlled manner in order to advance the prosthetic valve from the sheath. Alternatively, the power source can be operatively connected to the sheath in order to reciprocate the sheath longitudinally in the proximal direction relative to the prosthetic valve to deploy the prosthetic valve from the sheath.

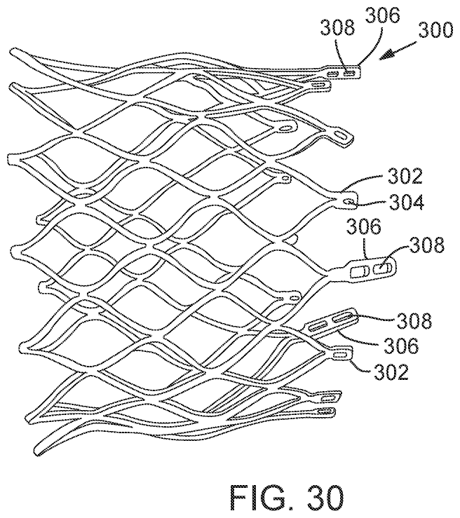

FIG. 30 shows another exemplary stent 300, for use in a prosthetic heart valve. For purposes of illustration, only the bare stent 300 is shown while the other components of the prosthetic valve, including the leaflets and the skirt, are omitted. However, in use the prosthetic valve can include leaflets 34a, 34b, 34c and a skirt 42 mounted to the stent 300, as described above in connection with the prosthetic valve 10. The stent 300 can have the same overall shape and configuration as the stent 12 of prosthetic valve 10 described above, except that all apices 302 at the outflow end of the stent 300 have respective apertures 304. The stent 300 can further comprise three commissure posts 306 (which are also “apices” herein) with eyelets 308, also at the outflow end. The delivery apparatus can engage the stent by wrapping suture loops around the apices at one end of the stent (e.g., the outflow end). In some embodiments, the stent can have notches, channels or other narrowed portions formed in or adjacent to the apices, for stably holding the suture loops against their respective apices. The frame 300 can be configured for delivery using any of the delivery apparatuses described herein. Additional embodiments of delivery apparatuses which can be used to deliver the stent 300 are described in U.S. Pat. No. 9,867,700, incorporated by reference above, and in U.S. Patent Application Publication No. 2015/0305867, which is incorporated herein by reference.

### Second Representative Embodiment

During deployment of self-expanding prosthetic heart valves such as the prosthetic valve 10, the prosthetic valve may be partially deployed or unsheathed from the delivery cylinder while the surgeon assesses placement of the prosthetic valve. If repositioning the prosthetic valve is desirable, the prosthetic valve may be partially or fully withdrawn back into the delivery cylinder or “recaptured” in order to reposition the prosthetic valve in the native annulus. Depending upon factors including the diameter of the prosthetic valve, the diameter of the delivery cylinder, the proportion of the overall length of the prosthetic valve that is outside the delivery cylinder before recapture is attempted, the number of times that recapture is attempted, etc., the frame of the prosthetic heart valve may fail to uniformly recollapse to a substantially cylindrical shape when recaptured.

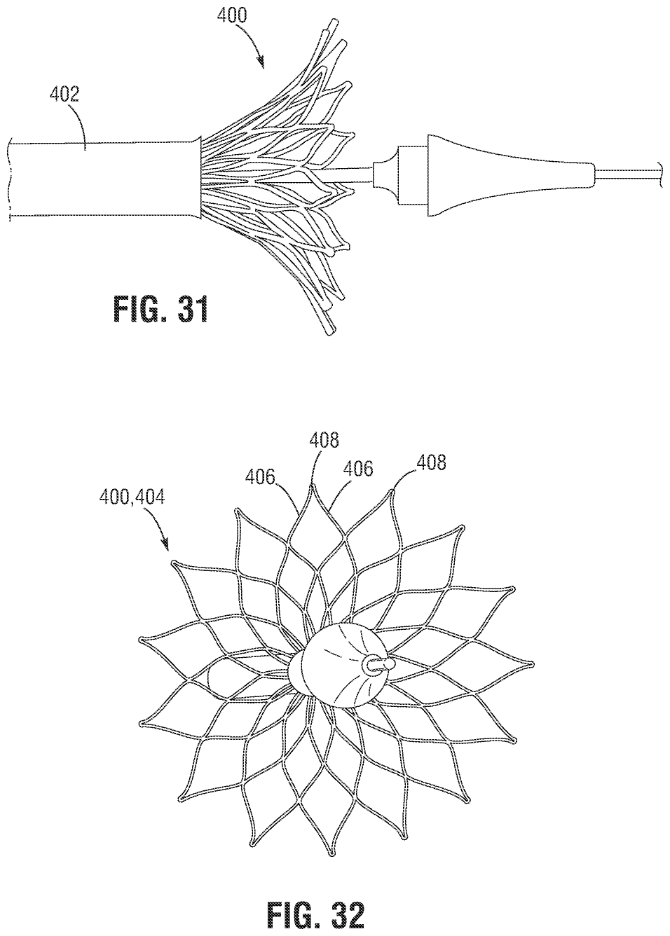

For example, FIG. 31 illustrates a self-expandable prosthetic valve frame 400 partially deployed from a delivery cylinder 402. In FIG. 31, about 80% of the overall length of the frame has been unsheathed, leaving 20% of the frame length within the delivery cylinder 402. FIGS. 32-35 illustrate recapture of the frame 400 after partial (e.g., 80%) deployment from the delivery cylinder. In FIGS. 31 and 32, the inflow end 404 of the frame forms a flared or cone shape extending distally from the delivery cylinder 402. In FIG. 32, the inflow end 404 of the frame has a circular or substantially circular shape. For example, in the illustrated configuration adjacent struts 406 can form a plurality of inflow apices 408 at junctions where adjacent struts 406 intersect. In the state illustrated in FIG. 32, the distance or diameter as measured between a pair of diametrically opposed apices 408 can be constant or substantially constant for any diametrically-opposed pair of apices around the circumference or perimeter of the inflow end 404.

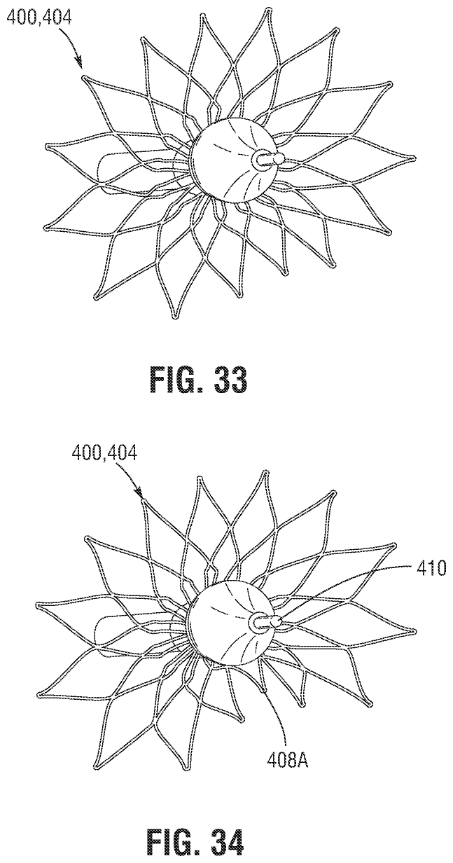

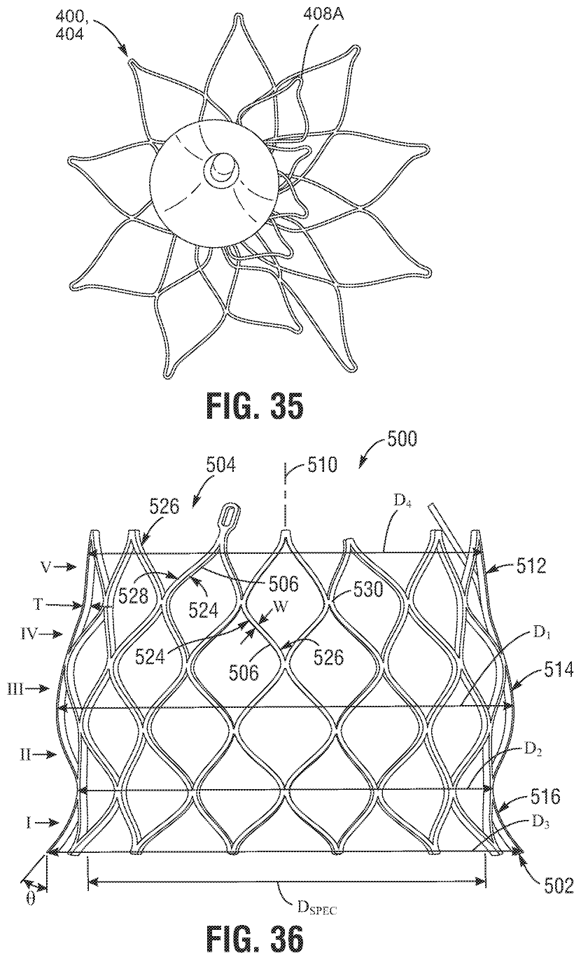

As the frame is withdrawn back into the delivery cylinder or “re-sheathed,” the inflow end desirably should maintain a circular or substantially circular profile with a constant or substantially constant diameter as measured at each apex 408 around the circumference of the inflow end. However, in certain instances, as the frame is withdrawn back into the delivery cylinder 402, one or more struts may bend, deform, buckle, or fold radially inward toward the longitudinal axis of the frame. This phenomenon is illustrated in FIG. 33, in which one or more struts in the lower right quadrant of the inflow end 404 begin to deform and the inflow end loses its circular or substantially circular shape. In FIG. 34, the deformation is more advanced, and an inflow apex 408A has deviated radially inwardly toward the guide wire 410. In FIG. 35, one or more struts formerly located in the lower right quadrant of FIGS. 32-34 have moved or buckled such that the inflow apex 408A and the adjacent struts have moved to the upper right quadrant of FIG. 35. This phenomenon forms a crease or infold in the frame, and is referred to herein as “infolding” or “invagination” of the frame. Such infolding can result in the need to discard the implant and insert a new prosthetic valve during an implantation procedure.

FIG. 36 illustrates another embodiment of a self-expandable frame 500 for a prosthetic heart valve configured to reduce the likelihood of an infolding event during recapture. For clarity, only the front half of the frame is shown. The frame 500 can comprise an inflow end 502 and an outflow end 504. The frame 500 can be formed from a plurality of angled strut members 506 arranged end-to-end to form a plurality of rows or rungs of strut members that extend circumferentially around the frame. For example, the frame 500 can comprise a first or lower row I of angled strut members 506 forming the inflow end 502 of the frame; a second row II of strut members above the first row; a third row III of strut members above the second row; a fourth row IV of strut members above the third row, and a fifth row V of strut members above the fourth row and forming the outflow end 504 of the frame. The struts 506 can be interconnected at nodes or junctions 530, which can demarcate the respective rows I-V. Traced in a direction along the longitudinal axis 510 of the frame, the struts 506 can combine to form generally sinusoidally-shaped members with apices formed by the junctions 530 so as to provide a mesh structure.

The frame can comprise a generally cylindrical “upper” or outflow end portion 512, an outwardly bowed or distended intermediate or belly portion 514, and an inwardly bowed “lower,” waist, or inflow end portion 516, similar to the frame of FIG. 1. The intermediate portion 514 can be sized and shaped to extend into the Valsalva sinuses in the root of the aorta to assist in anchoring the prosthetic valve, as in the embodiments described above.

When the frame is in its expanded state, the intermediate portion 514 can have a diameter D₁, the waist of the inflow end portion 516 can have a minimum diameter D₂, the inflow end 502 can have a diameter D₃, and the outflow end portion 512 can have a diameter D₄, where D₂ is less than D₁ and D₃, and D₄ is less than D₂. As with the embodiments described above, D₁ and D₃ can be greater than the diameter of the native annulus in which the prosthetic valve is to be implanted such that the frame assists in retaining the prosthetic valve at the implantation site. In certain embodiments, this configuration can also reduce or prevent paravalvular leakage.

The struts 506 can be made of a shape memory material, such as Nitinol or other nickel titanium alloys, that allow the prosthetic valve to be compressed to a reduced diameter for delivery in a delivery apparatus (such as described above) and then causes the prosthetic valve to expand to its functional size inside the patient's body when deployed from the delivery apparatus. In other embodiments, the frame can also comprise ductile materials such as nickel-chromium alloys or stainless steel, and can be configured for use with balloon-expandable valves.

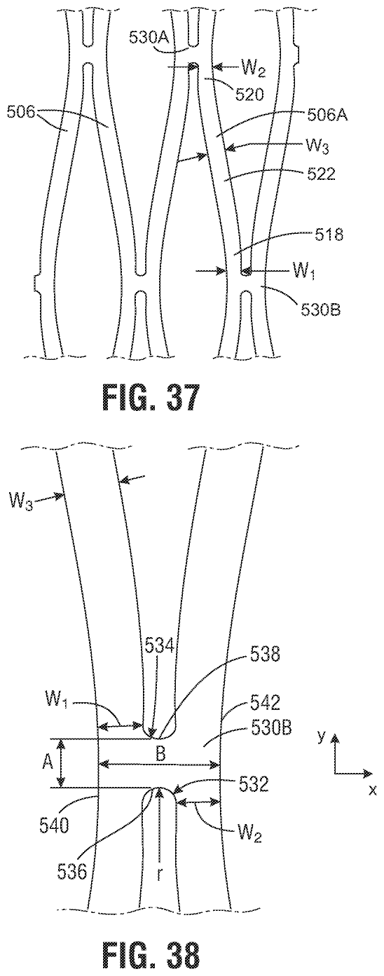

FIG. 37 illustrates a representative row of struts 506 of the frame 500 in a radially compressed state. Each of the struts 506 can comprise an inflow end portion 518, an outflow end portion 520, and an intermediate portion 522 extending between the inflow end portion and the outflow end portion. In certain embodiments, the dimensions of the struts 506 can vary along their lengths between the inflow and outflow ends of the struts. In certain embodiments, the dimensions of various portions of the struts of one strut row can be different than the dimensions of the corresponding portions of the struts in an adjacent strut row.

For example, the struts can comprise a thickness or width dimension measured generally in the plane of the curved exterior surface of the frame, referred to herein as the “strut width” W. Referring again to FIG. 36, each of the struts 506 can have a surface 524 oriented generally in the direction of the inflow end 502 when the frame is in the expanded state, a corresponding surface 526 on the opposite side of the strut and oriented generally in the direction of the outflow end 504 when the frame is in the expanded state. Each strut can further comprise an exterior surface 528 that is perpendicular to the surface 524 and to the surface 526. The thickness of the struts 506 as measured between the inflow surface 524 and the outflow surface 526 is referred to herein as the strut width W. Stated differently, the strut width W is the dimension of the exterior surface 528 of the strut 506 measured in a direction perpendicular to the strut's longitudinal axis. Each of the struts 506 can comprise a strut width as defined above. The corresponding dimension of the radially inward-facing surfaces of the strut members opposite the outer surfaces 528 can be the same or different as the strut widths of the outer surfaces 528, depending upon the particular characteristics desired.

Referring again to FIG. 36, the struts 506 can also have a wall thickness, radial thickness, or strut thickness T measured in the radial direction from the interior surfaces of the frame struts to the exterior surfaces 528 of the struts. In embodiments in which the frame 500 is formed from a tube (e.g., by laser-cutting), the struts of the frame can have a thickness T corresponding to the wall thickness of the tube from which the frame is cut. In other embodiments, the wall thickness of the tube and/or of the frame after laser cutting may be varied (e.g., by machining, reaming, etching, etc.), which can result in variation of the radial thickness of the struts.

Returning to FIG. 37, the struts 506 can define a first strut width W₁ at the inflow end portions 518, a second strut width W₂ at the outflow end portions 520, and a third strut width W₃ at the intermediate portions 522. These measurements are indicated on a representative strut member 506A extending between a junction 530A (e.g., an outflow junction of the strut 506A) and a junction 530B (e.g., an inflow junction of the strut 506A). FIG. 38 illustrates the junction 530B in greater detail. Referring to FIG. 38, in certain embodiments the junctions 530 between strut rows can define curved surfaces extending between adjacent strut members having radii r. For example, the representative junction 530B can comprise an inflow curved or concave surface 532 and an outflow curved or concave surface 534. In certain embodiments, each of the junctions 530 can comprise similar curved surfaces on the inflow and outflow aspects of the junction.

Referring to FIG. 38, in certain embodiments the strut width W₁ can be measured at or adjacent the edge of the outflow curved surface 534 of the junction 530B. In certain embodiments, the strut width W₂ can be measured at or adjacent the edge of the inflow curved surface 532 of the junction 530B. In certain embodiments, the strut widths W₁ and W₂ can be measured at the midpoints between the edge of the curved surface of the respective junction and the location where the strut width reaches the specified strut width W₃. In certain embodiments, the strut width W₁ can gradually increase to the strut width W₃ in a direction along the longitudinal axis of the strut. Likewise, at the opposite end of the strut, the strut width W₃ can gradually decrease to the strut width W₂. In other embodiments, some or all of the junctions need not comprise curved surfaces on the inflow and outflow aspects of the junctions, but may instead comprise straight surfaces, and/or convex surfaces.

In certain embodiments, the third strut width W₃ can be larger than the strut widths W₁ and W₂. In certain embodiments, the strut widths W₁ and W₂ can be the same or different, depending upon the particular characteristics desired. In certain embodiments, the strut widths W₁ and W₂ can be equal or substantially equal. As used herein, the strut widths W₁ and W₂ are substantially equal if their values differ by 10% or less. In certain embodiments, reducing the strut width at the junctions can advantageously reduce the radial force required to crimp the valve for delivery, as further described below.

In certain embodiments, the struts 506 of each of the strut rows I-V can be configured similarly to the representative strut member 506A. In certain embodiments, the strut width of the various portions of the struts can vary between rows. For example, in certain embodiments the struts of row I or rows I and II at the inflow end portion of the frame can comprise the varying strut width configuration shown in FIGS. 37 and 38, while the struts of the remaining rows can comprise a different configuration (e.g., a uniform strut width along the length of the struts, or other configurations).

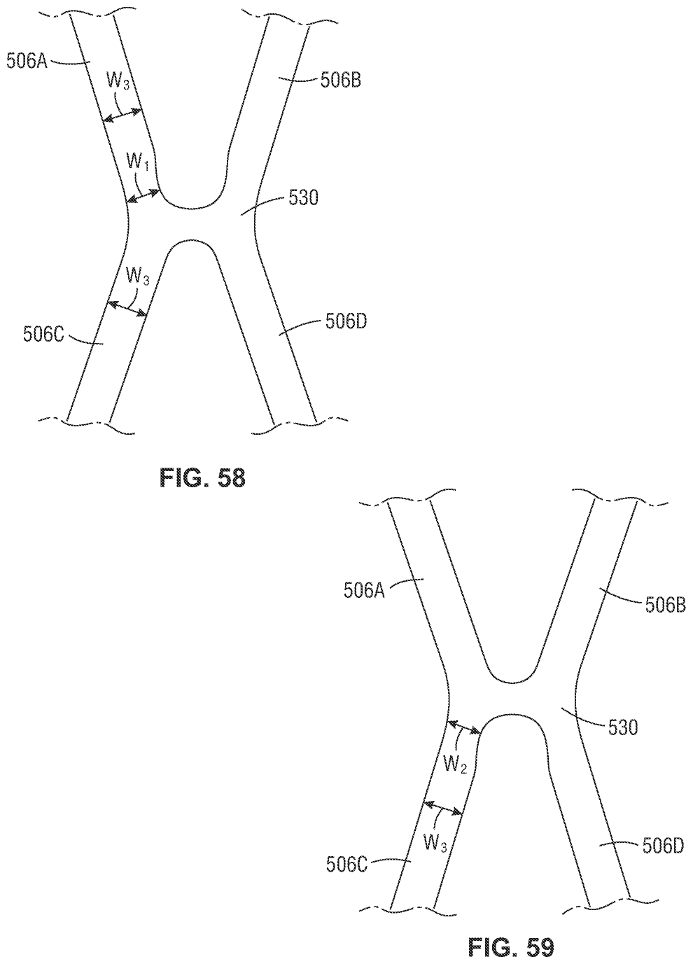

For example, FIG. 57 illustrates a junction 530 of another embodiment of the frame in which the struts 506A and 506B (e.g., on the outflow side of the junction) comprise the varying strut widths W₁, W₂, and W₃, and the struts 506C and 506D (e.g., on the inflow side of the junction) have a constant or substantially constant strut width along their lengths. In certain examples, the struts 506C and 506D can have a strut width less than the strut width W₃ (e.g., W₁ or W₂) as in FIG. 57, or equal or substantially equal to W₃, or greater than W₃. For example, FIG. 58 illustrates another configuration in which the struts 506C and 506D have the third strut width W₃ (or a different strut width) along substantially their entire lengths, while the struts 506A and 506B on the outflow side of the junction 530 have reduced strut widths W₁ at the junction. FIG. 59 illustrates the opposite configuration in which the struts 506C and 506D on the inflow side of the junction 530 have the varying strut widths at the junction, and the struts 506A and 506B have constant or substantially constant strut widths (e.g., W₃ or a different strut width) along their lengths.

Any two rows of struts coupled together at junctions such as the junctions 530 can have any of the varying or constant strut width configurations described herein. For example, in certain embodiments at least a portion of the struts of the frame can comprise a reduced strut width (e.g., W₁ or W₂) at at least one of their respective junctions, such as at their inflow junctions (e.g., junction 530B in FIG. 37), at their outflow junctions (e.g., junction 530A in FIG. 37), or both. In certain embodiments, the first row I of struts (FIG. 36) at the inflow end of the frame can comprise reduced strut widths at their inflow junctions, outflow junctions, or both. In certain embodiments, more than one row of struts, such as rows I and II, or rows I-III, or the row(s) configured to be deployed first from a delivery sheath, etc., can comprise reduced strut widths at one or both of the inflow and/or outflow junctions.

In certain embodiments, a length L of the strut members 506 can be from 4 mm to 6 mm. In certain embodiments, the length L of the strut members 506 can vary based upon the specified design diameter of the frame. For example, in certain examples a frame configured as described herein having a specified design diameter of 26 mm can have a strut length L of 4.33 mm. A frame having a specified diameter of 29 mm can have a strut length L of 4.79, and a frame having a specified design diameter of 32 mm can have a length L of 5.3 mm.

Returning to FIG. 38 the junction 530B can define inflow and outflow curved surfaces 532 and 534, as noted above. The curved surfaces 532 and 534 can both comprise the radius r, although in other embodiments the radii on the inflow and outflow sides of the junction can be different. The junction 530B can define a thickness dimension A extending along the y-axis between an apex 536 of the inflow curved surface 532 and an apex 538 of the outflow curved surface 534. The junction 530B can also define a junction width dimension B extending between longitudinally-oriented edges 540 and 542 of the junction.

The inventors have discovered that self-expandable frames for prosthetic heart valves including one or more of the parameters described herein individually and/or in various combinations can provide surprisingly superior performance, particularly when it comes to recapturing the prosthetic valve without infolding. The parameters and frame embodiments described herein can also provide improved performance with regard to radial force required to crimp the valve for delivery, and the “chronic” outward radial force applied by the frame to the surrounding anatomy once deployed at the treatment site.

For example, in certain embodiments a ratio of the strut widths W₁ and/or W₂ to the strut width W₃ can be 0.7 to 0.95, 0.75 to 0.95, 0.8 to 0.95, or less or equal than 0.90. In particular embodiments, the strut widths W₁ and W₂ can be 0.29 mm to 0.32 mm, and the strut width W₃ can be 0.33 mm to 0.37 mm. Reducing the strut width near the junctions 530 can reduce the radial force required to crimp the valve for delivery, while reducing the tendency of the frame to infold during recapture.

In certain embodiments, a ratio of the strut width W₃ to the strut thickness T can be 0.5 to 0.9, 0.6 to 0.85, 0.65 to 0.8, or greater than or equal to 0.65. In particular embodiments, the strut width W₃ can be 0.33 mm to 0.37 mm, and the strut thickness T can be 0.47 mm to 0.50 mm. A ratio of the strut width W₃ to the strut thickness T within the ranges given above can reduce the tendency of the frame to infold during recapture.

In certain embodiments, a ratio of the junction width B of the junctions 530 to the strut thickness T can be 1.4 to 3.2, such as 1.5 to 2.5, 1.5 to 2.1, or 1.5 to 2.0. In certain embodiments, the ratio of the junction width B to the strut thickness T can be greater than or equal to 1.5, or less than or equal to 2.1. In particular embodiments, the junction width B of the junctions 530 can be 0.7 mm to 1.5 mm, such as 0.8 mm to 1.0 mm, or 0.85 to 1.0 mm. In particular embodiments, the junction width B can be 0.91 mm, and the strut thickness T can be 0.47 mm to 0.50 mm. A ratio of the junction width B of the junctions to the strut thickness T within the ranges given above can provide radial force and crush resistance values within specifications for implantation in the heart, such as at the native aortic valve. For example, in certain embodiments, frames configured as described herein applied a maximum radial force of 145 N or less, such as 121 N or less, during crimping, and applied a chronic outward force of 30 N or more after expansion to the specified design diameter. These frames also displayed a crush resistance of 5 N to 8 N. In certain embodiments, the strut thickness T can have a relatively large effect on crush resistance and a relatively less pronounced effect on radial force, while the junction width B and/or the inflow and outflow strut widths W₁ and W₂ can have a relatively large effect on the radial force exerted by the compressed frame.

In certain embodiments, a ratio of the strut width W₃ to the junction width B can be 0.25 to 0.7, such as 0.3 to 0.6, 0.3 to 0.5, or 0.3 to 0.45. In certain embodiments, the ratio of the strut width W₃ to the junction width B can be greater than or equal to 0.3 or less than or equal to 0.45. In particular embodiments, the strut width W₃ can be 0.33 mm to 0.37 mm, and the junction width B can be 0.7 mm to 1.5 mm, such as 0.91 mm as noted above.

In certain embodiments, a ratio of the strut width W₁ and/or W₂ to the junction width B can be 0.2 to 0.5, such as 0.25 to 0.45 or 0.3 to 0.4. In certain embodiments, the ratio of the strut width W₁ and/or W₂ to the junction width B can be greater than or equal to 0.3 or less than or equal to 0.4. In particular embodiments, the strut width W₁ and/or W₂ can be 0.29 mm to 0.32 mm, and the junction width B can be 0.7 mm to 1.5 mm, such as 0.91 mm as noted above.

In certain embodiments, a ratio of the strut width W₂ of the outflow ends 520 of the struts to the radius r of the curved inflow surfaces 532 of the junctions can be 4.0 to 7.5, such as 4.1 to 7.1. A ratio of the strut width W₁ of the inflow ends 518 of the struts to the radius r of the curved outflow surface 534 can have similar values. In particular embodiments, the radii r of the curved surfaces 532 and/or 534 of the junctions 530 can be 0.04 mm to 0.08 mm, such as from 0.044 to 0.07 mm. Radii within these ranges can improve manufacturability and accuracy of the resulting surfaces, especially when using laser-cutting techniques where the diameter of the laser beam can be 0.04 mm. Larger junction radii can promote more even heat distribution through the metal of the frame during laser cutting, and can also reduce the formation of microcracks at the junctions from repeated crimping.

In certain embodiments, after the frame is cut from a tube, the frame can be electropolished, electrochemically polished, and/or etched in an etchant. These processes can alter the strut width, thickness, and/or junction radius parameters of the as-cut frame. Thus, in certain embodiments, the mass of the frame can be used to infer whether the strut width, strut thickness, and/or junction radius parameters are within specified ranges. For example, in certain embodiments of the frame 500 configured as described herein, the mass of the frame can vary from 800 to 1,100 mg, such as between 875 mg to 1,000 mg, or 950 mg to 990 mg. In particular embodiments, the mass of the frame 500 configured as described herein can be 975 mg.

In certain embodiments, the flared inflow end portion 516 can define an angle θ with respect to the longitudinal axis 510. In certain embodiments, configuring the inflow end portion such that the angle θ is within a specified range can reduce the tendency of the frame to infold during recapture. Keeping the angle θ within a specified range can also reduce the likelihood of the inflow end portion 516 contacting the bundle of His and interfering with electrical signaling in the heart post-implantation. In certain examples, an angle θ of less 30°, such as 25° or less, or 21° or less can provide sufficient flaring of the inflow end portion 516 to anchor the prosthetic valve in the native valve annulus, while reducing the risk of infolding during recapture and/or of contacting the His bundle. In particular embodiments, an angle θ of 21° in combination with locating the frame such that 5 mm of the inflow end portion 516 extends into the left ventricle can reduce the risk of contacting the His bundle.

Another parameter that can reduce the likelihood of infolding during recapture is the ratio of the inner diameter of the delivery cylinder to the diameter of the flared inflow end of the frame when partially deployed from the delivery cylinder. In certain embodiments, the frame can be configured to expand to a specified design diameter (also referred to as a specified diameter, a design diameter, or a deployment diameter). The particular specified design diameter of the prosthetic valve can correspond to, for example, the size and shape of the individual's anatomy into which the prosthetic valve is to be implanted. For self-expanding frames configured as described herein, the specified design diameter can be measured between the interior surfaces of the frame at the narrowest point of the inflow end portion 516. In other embodiments, the specified design diameter can be measured at the location of the smallest inner diameter of the frame anywhere along its length when the frame is expanded to its functional size. The specified design diameter D_{SPEC} of the frame 500 is illustrated in FIG. 36. For example, in certain embodiments prosthetic heart valves as configured herein can be provided with a specified design diameter of 23 mm, 26 mm, 28 mm, 29 mm, and 32 mm or larger.

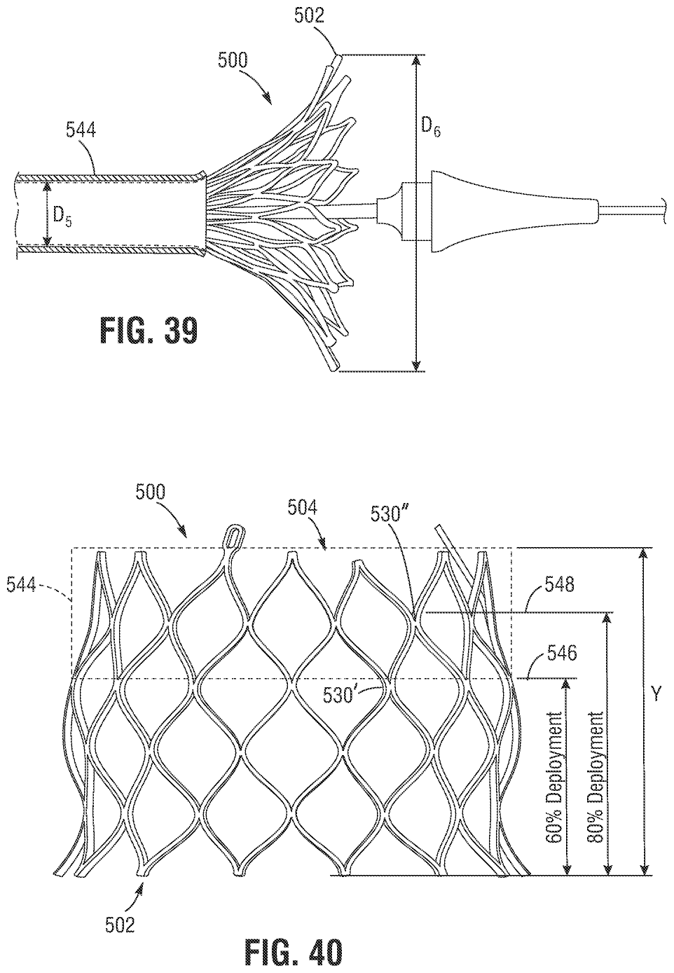

Typically, the specified design diameter of the prosthetic heart valve is selected to be slightly larger than the patient's native annulus (e.g., a 32 mm prosthetic valve may be selected to treat a patient with a 30 mm native annulus diameter). In certain embodiments, prosthetic heart valves with a specified design diameter of at least 29 mm or larger can be more prone to infolding during recapture after partial deployment. In certain embodiments, the ratio of the diameter of the inflow end of the partially expanded prosthetic valve to the inner diameter of the delivery cylinder can affect the tendency of the frame to infold or buckle during recapture. For example, FIG. 39 illustrates the frame 500 partially deployed from a delivery cylinder 544. For purposes of illustration, the portion of the frame 500 inside the delivery cylinder 544 is shown schematically in dashed lines. The delivery cylinder 544 can have an inner diameter D₅, and the flared inflow end 502 of the frame can have a diameter D₆. In certain embodiments, the inner diameter D₅ of the delivery cylinder 544 can be 6.35 mm for a prosthetic valve with a specified design diameter of 32 mm, 6.1 mm for a prosthetic valve with a specified design diameter of 29 mm, and 5.85 mm for a frame with a specified design diameter of 26 mm.

FIG. 40 illustrates the portions of the frame 500 that are located outside of the delivery cylinder when 60% of the overall length Y of the frame has been deployed, and when 80% of the overall length of the frame has been deployed. As used herein, the overall length Y of the frame 500 can be the length of the frame as measured between the inflow end 502 and the outflow end 504 when the frame is expanded to its specified design diameter. Thus, the latitudinal line 546 corresponds to 60% of the frame's overall length Y, and the latitudinal line 548 corresponds to 80% of the frame's overall length Y. In the illustrated embodiment, the latitudinal line 546 falls just above the junctions 530′ separating the strut row III from the strut row IV (FIG. 36). In other words, when 60% of the frame's overall length Y has been deployed from the delivery cylinder (shown schematically at 544 in FIG. 40), the junctions 530′ are just emerging from the distal end of the delivery cylinder. In the illustrated embodiment, the latitudinal line 548 falls just above the junctions 530″ separating the strut row IV from the strut row V (FIG. 36) Thus, as used herein, deploying 80% of the frame's overall length refers to the point at which the location 548 on the frame that is 80% of the distance between the inflow end 502 and the outflow end 504 when the frame is at its specified design diameter has been unsheathed or is outside (e.g., distal to) the delivery cylinder. In the illustrated configuration, 80% of the overall length Y of the frame has been deployed when the junctions 530″ emerge from the delivery cylinder 544.

Table 1 below provides exemplary dimensions for a 29 mm frame and a 32 mm frame configured similarly to the frame 500 of FIG. 36. These frame embodiments were tested and successfully recaptured into a delivery cylinder after being partially deployed with 80% of the frames' overall length unsheathed.

TABLE 1
Measurements of 29 mm and 32 mm Self-Expanding Frames

| | 29 mm | 32 mm |
|---|---|---|
| Measurement | Frame (mm) | Frame (mm) |
| Commissure Inner Diameter | 24.4 | 26.4 |
| Outflow Inner Diameter | 28.0 | 30.4 |
| Belly Outer Diameter D₁ | 32.0 | 35.2 |
| Waist Inner Diameter | 29.0 | 32.0 |
| Flare Outer Diameter D₃ | 33.5 | 36.4 |
| Height | 22.1 | 24.5 |
| Strut Width W₃ | 0.33 | 0.36 |
| Strut Width W₁ and W₂ | 0.29 | 0.31 |
| Junction Width B | 0.73 | 0.91 |
| Strut Thickness T | 0.44 | 0.48 |
| As-Cut Junction Radius | 0.064 | 0.070 |

In certain embodiments, when 80% of the overall length Y (FIG. 40) of the frame 500 has been deployed from the delivery cylinder 544, a ratio of the diameter D₆ of the inflow end 502 of the frame to the inner diameter D₅ of the delivery cylinder 544 can be greater than 4.5, such as 4.5 to 8.0, 5.0 to 7.0, 5.0 to 6.0, 5.2 to 6.2 or 5.5 to 6.0. In certain embodiments, for a frame with a specified design diameter of 29 mm or greater, the ratio of the diameter D₆ of the inflow end 502 of the frame to the inner diameter D₅ of the delivery cylinder 544 can be less than or equal to 6.0 when 80% of the overall length Y of the frame has been deployed from the delivery cylinder. In particular embodiments, the ratio of the diameter D₆ of the inflow end 502 to the inner diameter D₅ of the delivery cylinder 544 when 80% of the overall length of the frame is deployed from the delivery cylinder can be 5.7 to 6.0. In certain embodiments, a ratio of D₆ to D₅ within the ranges above can significantly reduce the likelihood of infolding during recapture, especially for larger design diameter valves where recapture is attempted with 80% or more of the overall length of the frame deployed from the delivery cylinder.

In certain embodiments, any of the delivery cylinders and/or apparatuses described herein can be configured to deliver other types of self-expanding implants, such as any of the prosthetic heart valve docking stations described below, stents, etc.

In addition to reducing the likelihood of infolding, the frame embodiments described herein also meet specified values for parameters including resistance to axial force (also known as crush force or crush resistance), radial force required during initial crimping of the valve, and the radial force applied by the frame against the surrounding tissue post-implantation (also known as the “chronic outward force”). FIG. 41 illustrates curves of force as a function of frame diameter for a frame having a specified design diameter of 32 mm and configured as described above. The natural, unconstrained diameter of the frame prior to crimping is about 37 mm. As the frame is crimped, the diameter and the force follow the upper curve 602 to a minimum diameter of 6-7 mm. At this diameter, the radial force exerted by the frame against the delivery cylinder (e.g., the force required to keep the frame radially constrained) is about 135 N. As the frame expands, the force/diameter relation follows the lower curve 604. Generally, self-expanding frames are oversized for the target annulus by 1-2 mm. Thus, at a diameter of 30 mm, the 32 mm frame exerts a chronic outward radial force of about 30 N. Existing 32 mm self-expanding frames exert a maximum radial force of about 145 N and a chronic outward radial force of 30 N.

Different embodiments of the self-expanding frames described herein can provide one or more significant advantages over existing self-expanding frames. For example, certain embodiments of the frames described herein can allow repeated partial deployment and recapture of frames having a relatively large specified design diameter without infolding or invagination. For example, self-expanding frames configured as described herein having a specified design diameter of 32 mm were successfully recaptured after deploying 80% of the frame's overall length, 90% of the frame's overall length, 95% of the frame's overall length, and 98% of the frame's overall length, all without infolding. The ability to repeatedly partially deploy and recapture a large diameter self-expanding valve can provide significant advantages when attempting to place a prosthetic valve in a relatively large anatomical structure, and can reduce the risk that a new prosthetic valve may be needed mid-procedure. The frames described herein also meet specifications for radial crimping force (e.g., 145 N or less), and chronic outward force when expanded at the treatment site (e.g., 28 N to 30 N, or more).

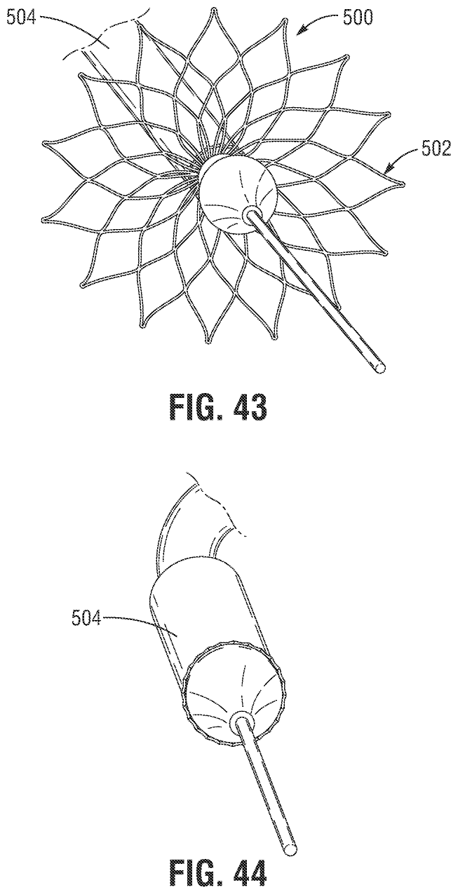

FIGS. 42-44 illustrate successful recapture of an embodiment of a frame 500 having a specified design diameter of 32 mm into a delivery sheath 544 having an inner diameter of 6.35 mm. In FIG. 42, 80% of the overall length of the frame is deployed from the delivery cylinder 544. Recapture is underway in FIG. 43, and FIG. 44 shows the frame fully recaptured within the delivery sheath 544 without infolding.

### Working Example 1

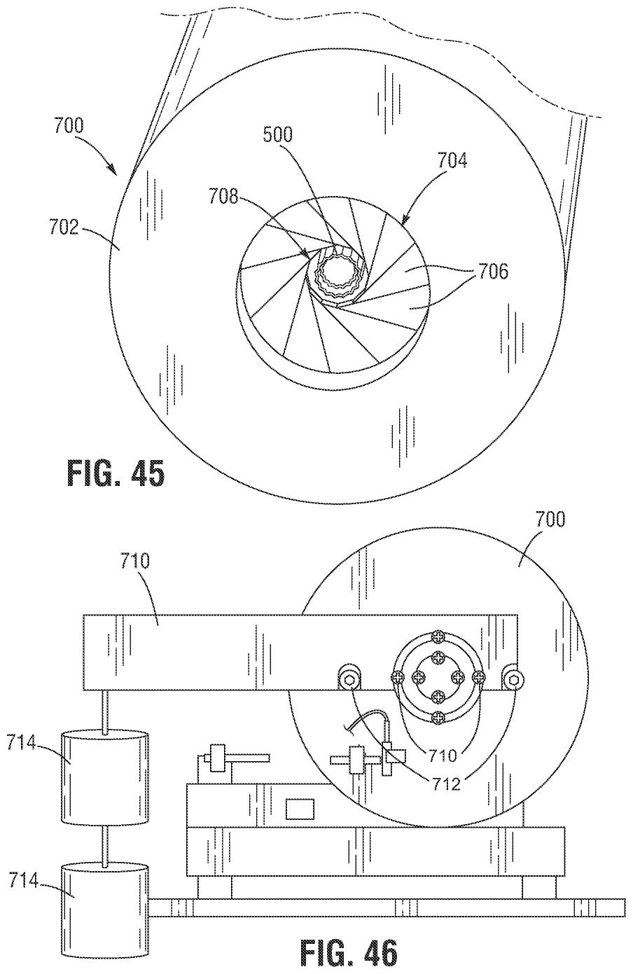

In a representative working example, testing and measurement of the radial force and chronic outward force of a frame 500 was conducted using a radial expansion force gauge apparatus 700 illustrated in FIG. 45. The apparatus 700 includes a main body 702 and an iris assembly 704 comprising a plurality of wedge-shaped members or mandrels 706. The members 706 define a central opening or lumen 708 configured to receive the frame 500. The members 706 can be actuated to controllably and uniformly reduce the diameter of the lumen 708 along the length of the frame in order to radially collapse the frame.

The tester apparatus 700 was calibrated by, for example, checking that the apparatus 700 was level, and that the apparatus was at the specified temperature. In the present example, the test was conducted at 37° C. To calibrate the temperature readout of the apparatus 700, a calibrated temperature sensor such as a thermocouple and/or a calibrated digital thermometer was inserted into the environmental chamber head of the apparatus, such as into the lumen 708, to a depth of 50.8 mm to 76.2 mm. After a specified period of time (e.g., 5 minutes), a temperature compensation value was entered such that the temperature readout of the apparatus matched the temperature sensor.

To calibrate the diameter of the iris assembly 704, a 6 mm diameter gauge pin was inserted at least 40 mm into the lumen 708 and a calibration routine was run. Next, a 40 mm diameter gauge pin was inserted at least 40 mm into the iris assembly and a calibration routine was run. To calibrate load cell(s) of the apparatus 700, a calibration yoke 710 was attached or hung from specified screws 712 on the apparatus 700 when the screws 712 were level (e.g., at a diameter of 10 mm). Weights 714 of varying mass were then attached to the yoke 710 to calibrate the load cells.

Friction of the various elements of the iris assembly 704 was then checked. In the present example, the measured friction was within ±1.5 N radial force.

During the test, a preset routine was selected for the 32 mm frame starting at a first diameter of 37 mm, and radially contracting at a rate of 0.5 mm/s to a second diameter of 6.35 mm (corresponding to the inner diameter of the delivery cylinder). The frame was inserted into the lumen 708 and allowed to acclimatize for two minutes before the test was initiated. The radial force exerted by the compressed frame was then measured, the results of which are shown in the graph of radial force versus diameter illustrated in FIG. 41.

### Third Representative Example

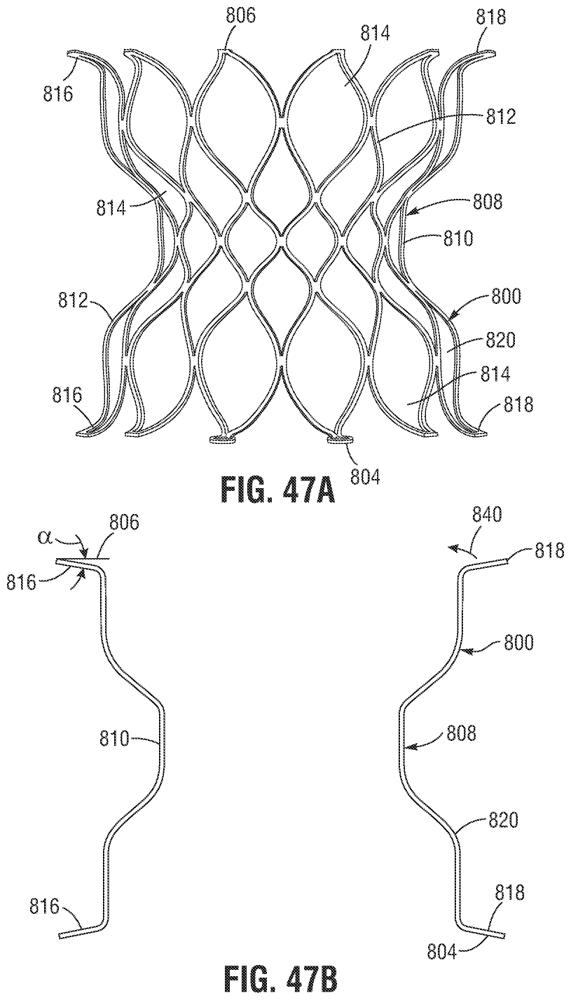

The varied strut widths, junction widths, junction radii, etc., described above can also be implemented on frames for other types of prosthetic implants, such as docking stations or systems configured to receive a prosthetic heart valve. One representative example of such a docking station is illustrated in FIGS. 47A-51.

FIGS. 47A and 47B illustrate an exemplary embodiment of a frame 800 or body of a docking station 802. The frame 800 or body can take a wide variety of different forms and FIGS. 47A and 47B illustrate just one of the many possible configurations. In the example illustrated by FIGS. 47A and 47B, the docking station 802 has a relatively wider proximal inflow end 804 and distal outflow end 806, and a relatively narrower portion 808 that forms the seat 810 in between the ends 804, 806. In the example illustrated by FIGS. 47A and 47B, the frame 800 of the docking station 802 is preferably a wide stent comprised of a plurality of metal struts 812 that form cells 814. In the example of FIGS. 47A and 47B, the frame 800 has a generally hourglass-shape that has a narrow portion 808, which forms the valve seat 810 when covered by an impermeable material, in between the proximal and distal ends 804, 806. As described below, the prosthetic valve expands in the narrow portion 808, which forms the valve seat 810.

FIGS. 47A and 47B illustrate the frame 800 in its unconstrained, expanded condition. In this exemplary embodiment, the retaining portions 816 comprise ends 818 of the metal struts 812 at the proximal and distal ends 804, 806. The sealing portion 820 is between the retaining portions 816 and the waist 808. In the unconstrained condition, the retaining portions 816 extend generally radially outward and are radially outward of the sealing portion 820. The frame 800 can be radially compressed for delivery and expansion by a catheter. The docking station can be made from a resilient or compliant material to accommodate large variations in the anatomy. For example, the docking station can be made from a highly flexible metal, metal alloy, polymer, or an open cell foam. An example of a highly resilient metal is nitinol, but other metals and highly resilient or compliant non-metal materials can be used. The docking station 802 may be self-expanding, manually expandable (e.g., expandable via balloon), or mechanically expandable. A self-expanding docking station 802 may be made of a shape memory material such as, for example, nitinol.

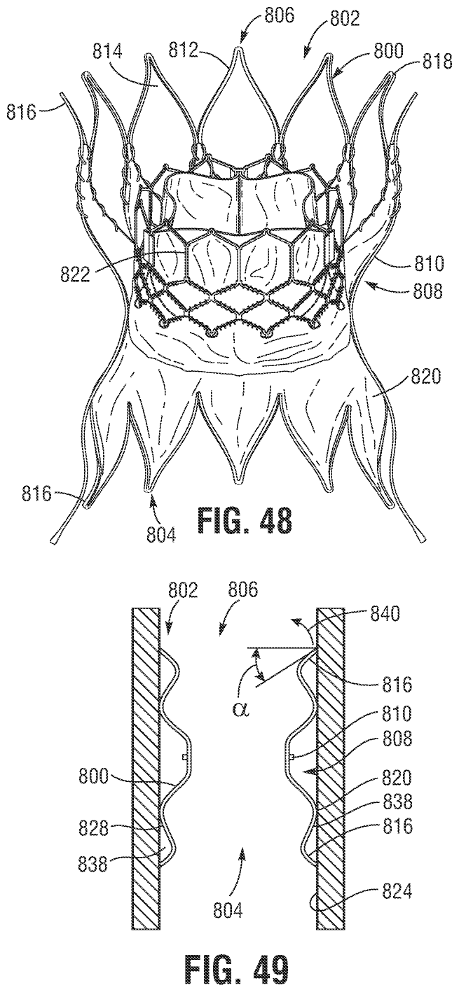

FIG. 48 illustrates a prosthetic valve 822 implanted in the frame 800. The valve 822, when docked within the docking station, can optionally expand around either side of the valve seat slightly. This aspect, sometimes referred to as a “dogbone” (e.g., because of the shape it forms around the valve seat or band), can also help hold the valve in place. In certain embodiments, the prosthetic valve 822 can be the SAPIEN® 3 balloon-expandable transcatheter heart valve available from Edwards Lifesciences Corporation. Details regarding the SAPIEN® 3 transcatheter heart valve can be found in U.S. Pat. No. 9,393,110, which is incorporated herein by reference. Additional embodiments of balloon expandable prosthetic heart valves that can be used in combination with the docking system 802 can be found in U.S. Publication No. 2018/0028310, which is incorporated herein by reference. The docking system 802 can also be used in combination with mechanically-expandable prosthetic valves. Representative examples of mechanically-expandable prosthetic valves can be found in U.S. Publication No. 2018/0153689 and U.S. Publication No. 2019/01056153, which are incorporated herein by reference.

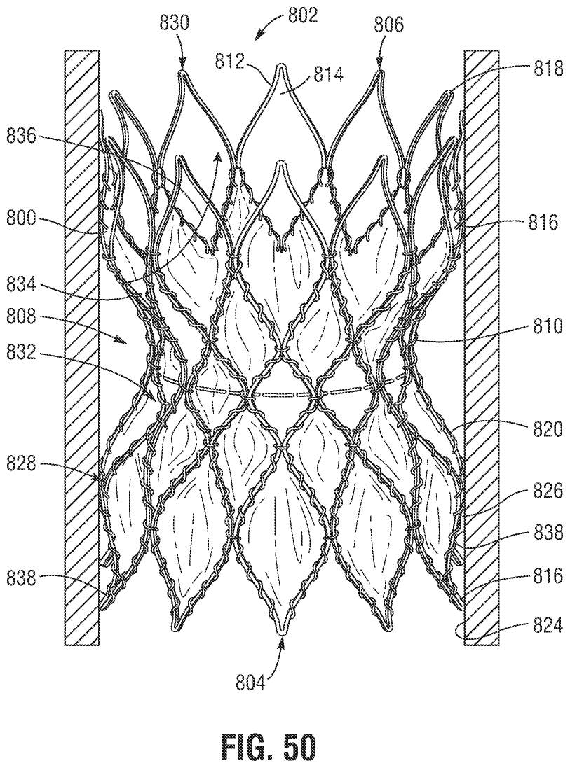

FIGS. 49 and 50 illustrate the docking station 802 of FIG. 47A implanted in the circulatory system, such as in the pulmonary artery. The sealing portions 820 provide a seal between the docking station 802 and an interior surface 824 of the circulatory system. In the example of FIGS. 49 and 50, the sealing portion 820 is formed by providing an impermeable material 826 (see FIG. 50) over the frame 800 or a portion thereof. In particular, the sealing portion 820 can comprise the lower, rounded, radially outward extending portion 828 of the frame 800. In an exemplary embodiment, the impermeable material 826 extends from at least the portion 828 of the frame 800 to the valve seat 810. This makes the docking station impermeable from the sealing portion 820 to the valve seal 810. As such, all blood flowing in the inflow direction 804 toward the outflow direction 806 is directed to the valve seat 810 (and valve 822 once installed or deployed in the valve seat).

In a preferred embodiment of a docking station 802, the inflow portion has walls that are impermeable to blood, but the outflow portion walls are relatively open. In one approach, the inflow end portion 804, the mid-section 808, and a portion of the outflow end portion 806 are covered with a blood-impermeable fabric 826, which may be sewn onto the stent or otherwise attached by a method known in the art. The impermeability of the inflow portion of the stent helps to funnel blood into the docking station 802 and ultimately flow through the valve that is to be expanded and secured within the docking station 802.

From another perspective, this embodiment of a docking station is designed to seal at the proximal inflow section 828 to create a conduit for blood flow. The distal outflow section, however, is generally left open, thereby allowing the docking station 802 to be placed higher in the pulmonary artery without restricting blood flow. For example, the permeable portion 830 may extend into the branch of the pulmonary artery and not impede or not significantly impede the flow of blood past the branch. In one embodiment, blood-impermeable cloth, such as a PET cloth for example, or other material covers the proximal inflow section, but the covering does not cover any or at least a portion of the distal outflow section 806. As one non-limiting example, when the docking station 802 is placed in the pulmonary artery, which is a large vessel, the significant volume of blood flowing through the artery is funneled into the valve 822 by the cloth covering 826. The cloth 826 is fluid impermeable so that blood cannot pass through. Again, a variety of other biocompatible covering materials may be used such as, for example, foam or a fabric that is treated with a coating that is impermeable to blood, polyester, or a processed biological material, such as pericardium.

In the example illustrated by FIG. 50, more of the docking station frame 800 is provided with the impermeable material 826, forming a relatively large impermeable portion 832. In the example illustrated by FIG. 50, the impermeable portion 830 extends from the inflow end 804 and stops one row of cells 814 before the outflow end. As such, the most distal row of cells 814 form a permeable portion 830. However, more rows of cells 814 can be uncovered by the impermeable material to form a larger permeable portion. The permeable portion 830 allows blood to flow into and out of the area 834 as indicated by arrows 836. With respect to the inflow end 804, it should be noted that since the cells 814 are generally diamond shaped, blood is able to flow between the docking station 802 and the surface 824, until the sealing portion 820 is reached. That is, blood can flow into and out of the areas 838 in one exemplary embodiment.

The valve seat 810 can provide a supporting surface for implanting or deploying a valve 822 in the docking station 802. The retaining portions 816 can retain the docking station 802 at the implantation position or deployment site in the circulatory system. The illustrated retaining portions have an outwardly curving flare that helps secure the docking station 802 within the artery. “Outwardly” as used herein means extending away from the central longitudinal axis of the docking station. As can be seen in FIG. 49, when the docking station 802 is compressed by the inside surface 824, the retaining portions 816 engage the surface 824 at an angle α (normal to the surface to the tangent of the midpoint of the surface of the retaining portion 816) that can be between 30 and 60 degrees, such as about 45 degrees, rather than extending substantially radially outward (e.g., a is 0 to 20 degrees or about 10 degrees) as in the uncompressed condition (see FIG. 47B). This inward bending of the retaining portions 816 as indicated by arrow 840 acts to retain the docking station 802 in the circulatory system. The retaining portions 816 are at the wider inflow end portion 804 and outflow end portion 806 and press against the inner surface 824. The flared retaining portions 816 engage into the surrounding anatomy in the circulatory system, such as the pulmonic space. In one exemplary embodiment, the flares serve as a stop, which locks the device in place. When an axial force is applied to the docking station 802, the flared retaining portions 816 are pushed by the force into the surrounding tissue to resist migration of the stent as described in more detail below. In a specific embodiment, the docking station generally has an hourglass shape, with wider distal and proximal end portions that have the flared retaining portion and a narrow, banded waist in between the ends, into which the valve is expanded.

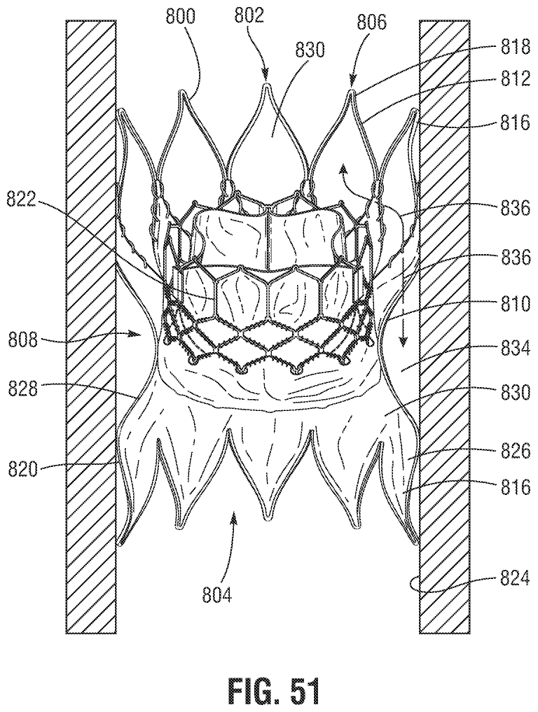

FIG. 51 illustrates the docking station 802 deployed in the circulatory system and a valve 822 deployed in the docking station 802. After the docking station 802 is deployed, the valve 822 is in a compressed form and is introduced into the valve seat 810 of the docking station 802. The valve 822 is expanded in the docking station, such that the valve 822 engages the valve seat 810. In the example illustrated by FIG. 51, the docking station 802 is longer than the valve. However, in one embodiment, the docking station 802 may be the same length or shorter than the length of the valve 822.

The prosthetic valve 822 may be expanded at the site of the docking station via means including balloon or mechanical expansion or by self-expansion. When the valve 822 is expanded, it nests in the valve seat of the docking station 802. In one embodiment, the banded waist is slightly elastic and exerts an elastic force against the prosthetic valve 822, to help hold the prosthetic valve in place.

As noted above, any of the struts of the docking station frame 800 can comprise the varied strut widths, junction widths, junction radii, etc., described above. For example, struts of any of the various strut rows of the frame 800 can comprise the narrower or tapering strut width adjacent the junctions, and the wider intermediate strut width at portions located between junctions according to any of the ratios described herein. The width of the junctions can also be greater than the intermediate strut width according to any of the ratios described herein. A ratio of a diameter of the inflow end of the docking station 800 to the inner diameter of a delivery cylinder from which the docking station is deployed can also be less than or equal to 6.0, as described above. Any or all of these features individually and/or in combination can reduce the tendency of the docking station frame 800 to infold during deployment and recapture. Additional details regarding the docking station 800 can be found in U.S. Publication No. 2017/0231756, which is incorporated herein by reference.

### Fourth Representative Embodiment

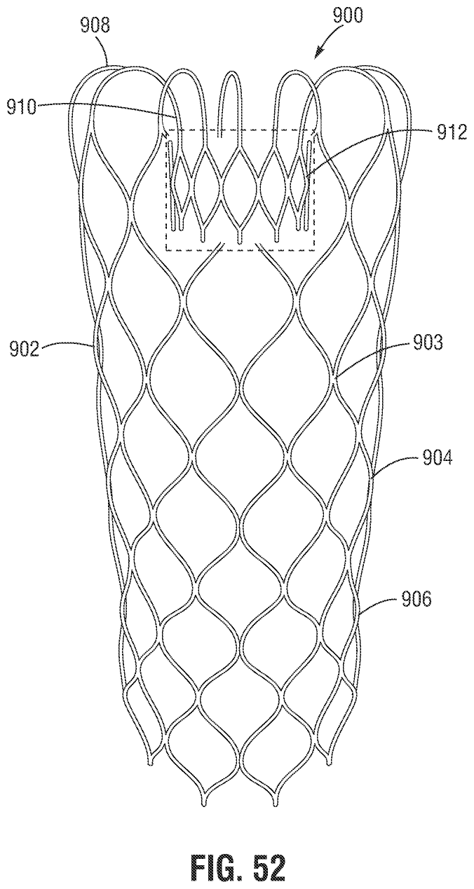

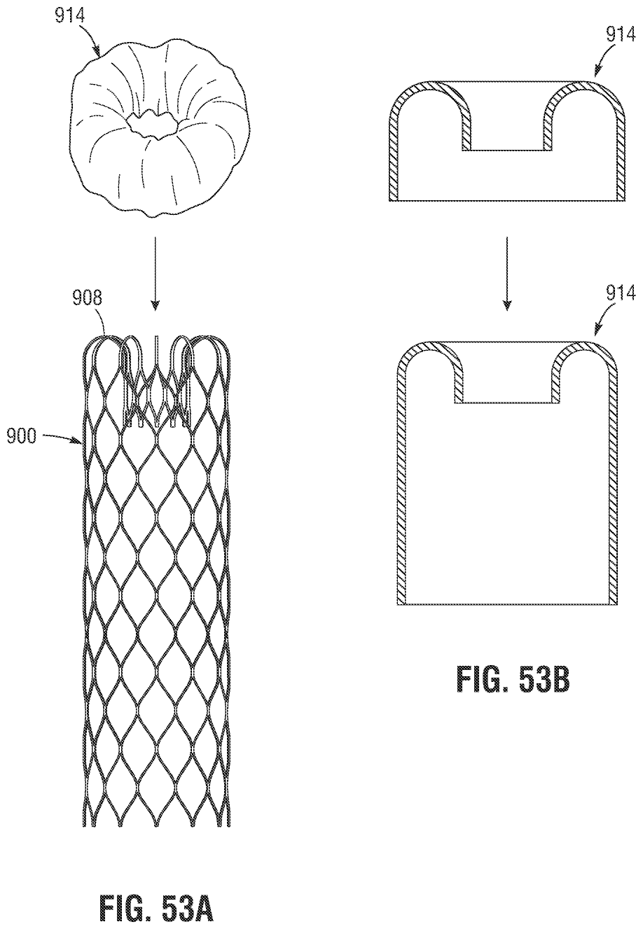

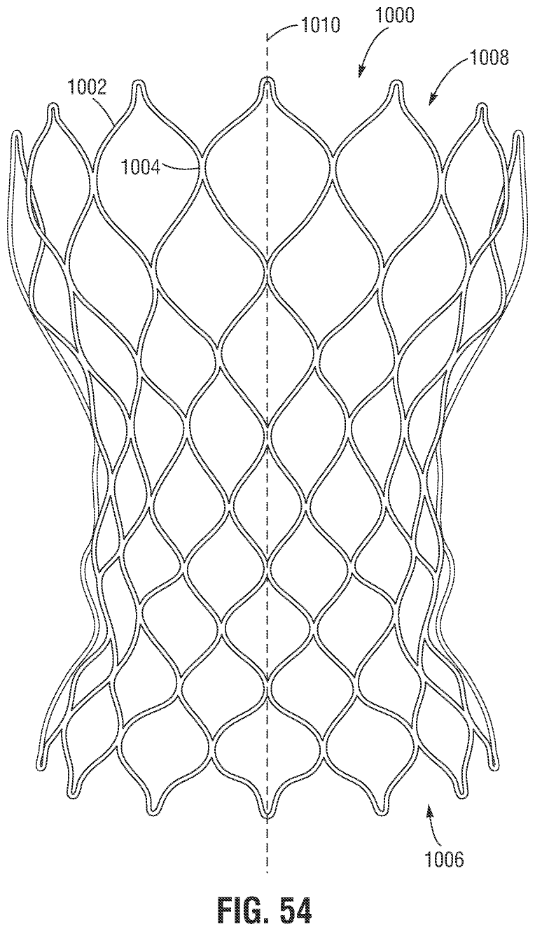

FIGS. 52-53B illustrate another embodiment of a docking system 900 configured to receive a prosthetic heart valve, and which can comprise any of the varied strut widths, junction widths, junction radii, etc., described herein in various combinations. FIG. 52 illustrates an exemplary embodiment of a frame 902 of the docking system 900 comprising a plurality of strut members 903 arranged in a lattice pattern. In certain embodiments, the struts 903 can vary in length and/or thickness, as described in U.S. Publication No. 2019/0000615, which is incorporated herein by reference. The frame 902 can take a wide variety of different forms and FIG. 52 illustrates just one of the many possible configurations. In certain embodiments, the frame 902 can comprise an elastic or superelastic material or metal, such as a nitinol.

The frame 902 can comprise a retaining portion 904 comprising an annular outer portion or wall 906 having a toroidal end surface 908. A shape set (e.g., a programmed shape of a shape memory material) of the annular outer portion 906 can bias the wall 906 radially outward into contact with/against an interior surface of a vessel (e.g., the aorta) to retain the docking station 900 and a prosthetic valve received therein at the implantation position. The frame 902 can further comprise legs or members 910 which extend from the perimeter of the frame into the lumen for supporting a valve seat 912, which can be configured to receive a prosthetic heart valve such as any of the prosthetic heart valves described herein.

Referring to FIGS. 53A and 53B, in certain embodiments the frame 902 can comprise a sealing material or covering 914 disposed on the end 908 of the frame to effect a seal between a prosthetic heart valve received in the valve seat 912 and the surrounding anatomy. The covering 914 can be configured as a cylinder rolled partially backward on itself. The cover 914 can comprise one or more sheets of polyethylene terephthalate (PET), polytetrafluoroethylene (PTFE), expanded PTFE (ePTFE), or any other polymers or biocompatible materials. In certain embodiments, the cover 914 can comprise a woven or knitted fabric comprising any of the materials above. Further details of the cover 914 can be found in U.S. Publication No. 2019/0000615 incorporated by reference above.

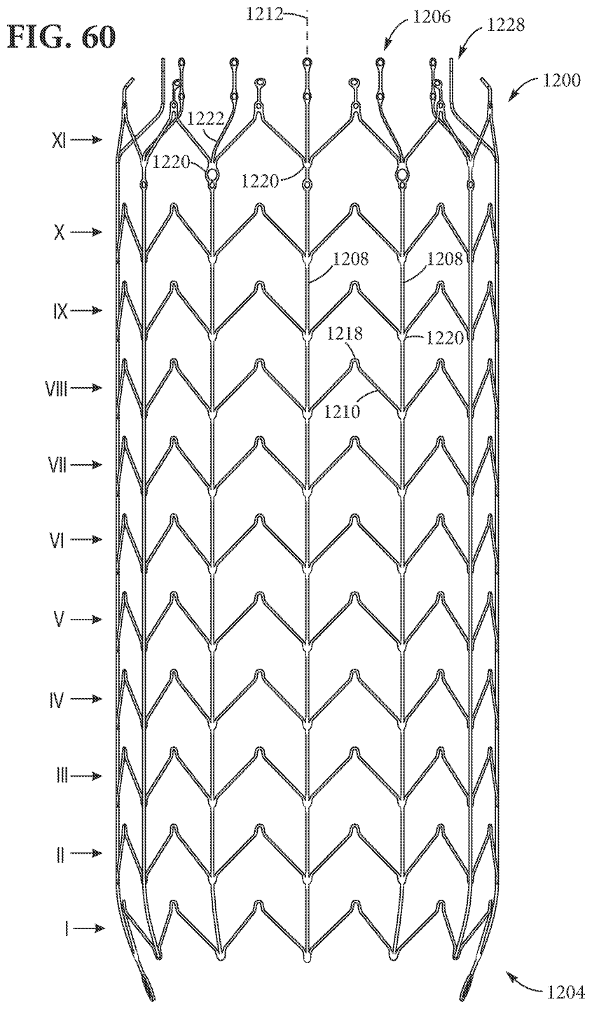

FIG. 60 illustrates another embodiment of a frame 1200 of a docking system configured to receive a prosthetic heart valve, and which can also comprise any of the varied strut widths, junction widths, junction radii, etc., described herein. The frame 1200 can comprise an inflow end portion 1204 and an outflow end portion 1206. The frame 1200 can comprise a plurality of longitudinal strut members 1208 circumferentially spaced apart from each other around the frame 1200. The frame can further comprise a plurality of rows of struts 1210 arranged alternatingly in a zig-zag pattern. The rows of struts 1210 can be axially spaced apart from each other along the longitudinal axis 1212 of the frame. For example, in the illustrated embodiment the frame 1200 can comprise eleven rows I-XI of struts 1210, with the first row I located at the inflow end portion 1204 and the eleventh row XI located at the outflow end portion 1206. The struts 1210 are arranged such that first end portions of the struts are coupled to longitudinal strut members 1208 at junctions 1220, and second end portions of the struts are coupled to second end portions of adjacent struts 1210 to form “free” apices 1218. The outflow end portion 1206 can comprise a plurality of struts 1222 coupled to the junctions 1220 of the eleventh row XI of struts. The struts 1222 can extend in a downstream direction, and can be angled radially inwardly toward the longitudinal axis 1212 to define a valve-receiving portion or valve seat generally indicated at 1228, which can be coaxial with the frame 1200 and configured to receive a prosthetic valve. Further details of the docking station frame 1200 can be found in U.S. Provisional Application No. 63/073,643, which is incorporated herein by reference.

Any of the struts of the docking station frame 900 and/or 1200 can comprise the varied strut widths, junction widths, junction radii, etc., described above. For example, struts of any of the various strut rows of the frames 900 and/or 1200 can comprise the narrower or tapering strut width adjacent the junctions, and the wider intermediate strut width at portions located between junctions according to any of the ratios described herein. The width of the junctions can also be greater than the intermediate strut width according to any of the ratios described herein. A ratio of a diameter of the inflow end of the docking station frame 900 and/or 1200 to the inner diameter of a delivery cylinder from which the docking station is deployed can also be less than or equal to 6.0, as described above. The struts and junctions can also be configured such that ratios of the various strut widths to the radii of the curved surfaces of the junctions fall within any of the ranges described herein. Any or all of these features individually and/or in combination can reduce the tendency of the docking station frames 900 and 1200 to infold during deployment and recapture.

### Fifth Representative Embodiment

FIG. 54 illustrates another embodiment of a frame 1000 of a prosthetic heart valve comprising a plurality of angled struts 1002 coupled together at junctions 1004. The frame 1000 can be configured as a self-expanding frame comprising any of the self-expandable materials described herein, and can be movable between a collapsed delivery configuration and an expanded functional configuration. The frame can have an inflow end 1006 and an outflow end 1008. The diameter of the frame 1000 can vary along its longitudinal axis 1010, as shown.

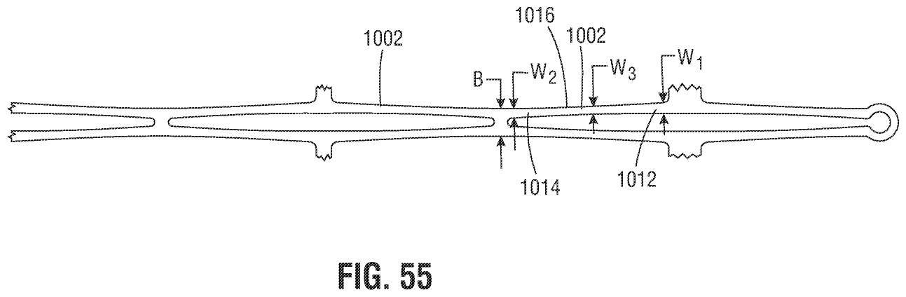

FIG. 55 illustrates a selected portion of the frame 1000. The struts 1002 can comprise first or inflow end portions 1012 and second or outflow end portions 1014 adjacent respective junctions 1004. The struts can further comprise third or intermediate portions 1016 located between the inflow end portions 1012 and the outflow end portions 1014. The inflow end portions 1012 can have a strut width W₁, and the outflow end portions 1014 can have a strut width W₂. Although the struts are shown narrowing from the junctions toward the middle of the struts, in certain embodiments the intermediate portions 1016 can have a strut width W₃ that is greater than the strut widths W₁ and W₂ as described above. The strut widths W₁, W₂, and W₃ can have any of the values and ratios described herein.

The junctions 1004 can also comprise a junction width B. The junction width B can be larger than the intermediate strut width W₃, as described above. A ratio of the intermediate strut width W₃ to the junction width B can be any of the ratios described herein. The struts 1002 can also have a strut thickness configured according to any of the dimensions and ratios described herein. In certain embodiments, the frame 1000 can be configured such that when 80% of an overall length of the frame is deployed from a delivery cylinder, a ratio of the diameter of the flared inflow end (or outflow end) of the frame to an inner diameter of the delivery cylinder is 6.0 or less. The struts and junctions can also be configured such that ratios of the various strut widths to the radii of the curved surfaces of the junctions fall within any of the ranges described herein. In certain embodiments, these features alone and/or in various combinations can reduce the tendency of the frame 1000 to infold during loading, deployment, and/or recapture of the prosthetic valve.

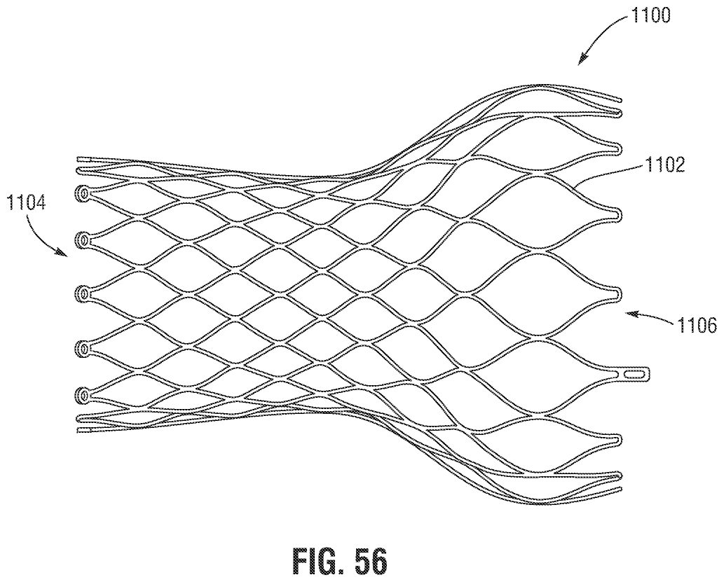

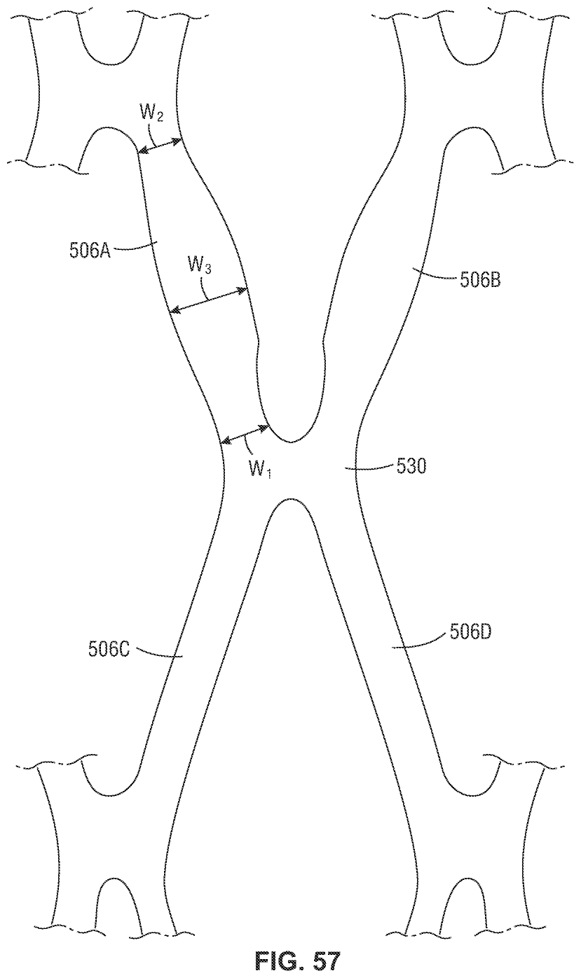

FIG. 56 illustrates another embodiment of a frame 1100 for a self-expanding prosthetic heart valve comprising a plurality of angled strut members 1102, an inflow end 1104 and an outflow end 1106. The frame struts 1102 can be configured according to any of the embodiments described herein to reduce the tendency of the frame 1100 to infold during loading, deployment, and/or recapture of the prosthetic valve.

### Sixth Representative Embodiment

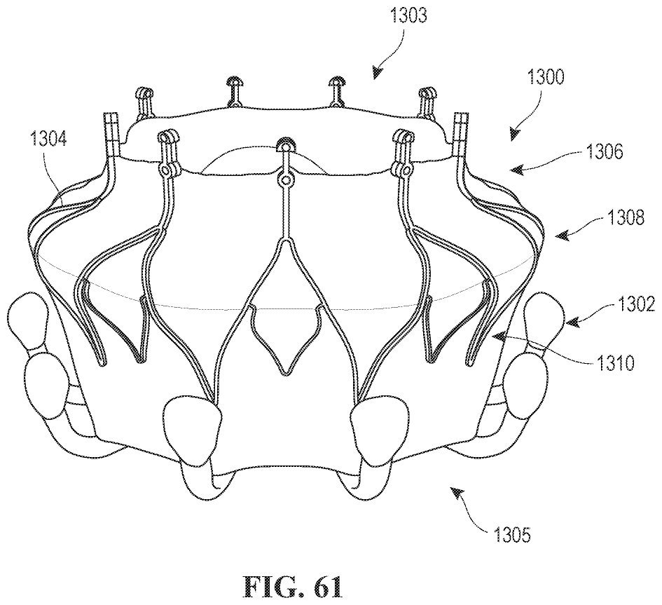

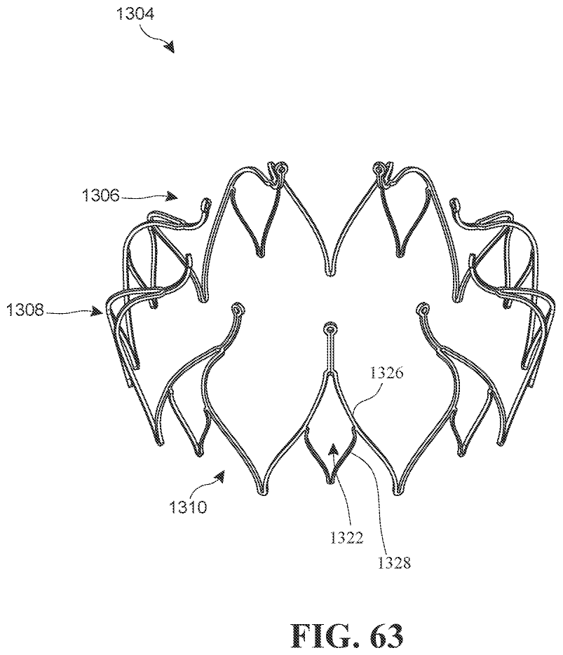

Any of the frame strut configurations, junction width configurations, etc., described herein can also be implemented in combination with prosthetic devices including multiple frames, or multiple layers of frames, such as inner and outer frames. Additionally, for prosthetic implants where the outflow end is deployed from the delivery sheath first, the varying strut width concepts described herein can be implemented on the struts at least at the outflow end of the frame. Such implants can include prosthetic heart valves configured for implantation (e.g., trans-septally) in the native mitral valve. For example, FIGS. 61-64 illustrate another embodiment of a self-expanding prosthetic implant configured as a prosthetic heart valve 1300 configured for implantation in the native mitral valve. Referring to FIG. 61, the prosthetic heart valve 1300 can comprise an inner frame 1302 and an outer frame 1304. The prosthetic heart valve 1300 can also have an inflow end 1303 and an outflow end 1305. The outer frame 1304 can have an upper region 1306, an intermediate region 1308, and a lower region 1310. In some situations, such as those in which the prosthesis 1300 is positioned within a native mitral valve, the upper region 1306 can be generally positioned supra-annularly, the intermediate region 1308 can be generally positioned intra-annularly, and the lower region 1310 can be positioned sub-annularly. The outer frame 1302 is depicted in isolation in FIG. 63.

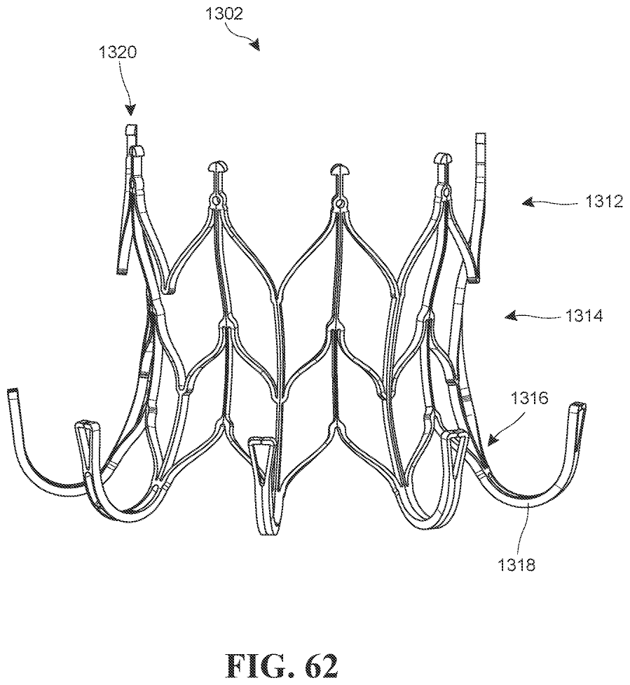

A representative embodiment of the inner frame 1302 is illustrated in FIG. 62. The inner frame 1302 can include an upper region 1312, an intermediate region 1314, and a lower region 1316. As shown, the intermediate region 1314 can have a smaller diameter than the upper region 1312, the lower region 1316, or both. This can form an hourglass shape wherein the intermediate region 1314 is thinner in diameter than both the upper region 1312 and the lower region 1316. In some embodiments, the upper region 1312 and the lower region 1316 can have approximately the same diameter. In certain embodiments, the inner frame 1302 can comprise an inner frame anchoring feature including a plurality of individual anchor members 1318 extending curvilinearly from the lower region 1316 in the radially outward direction and comprising tips configured to contact/engage intraluminal tissue upon implantation in the native mitral valve. The inner frame 1302 can also comprise a plurality of locking tabs 1320 configured to couple the prosthetic valve to a delivery system.

Referring to FIG. 63, the outer frame 1304 can include a plurality of struts with at least some of the struts forming cells 1322. The cells 1322 can have an irregular octagonal shape such as a “teardrop” shape, and can be formed via a combination of struts. As shown in the illustrated embodiment, the upper portion of cells 1322 can be formed from a set of circumferentially-expansible struts 1326 having a zig-zag or undulating shape forming a repeating “V” shape. The circumferentially-expansible struts 1326 can be inclined or curved radially outwardly away from the longitudinal axis of the prosthesis 100 such that an upper portion of the struts 1326 are positioned closer to the longitudinal axis of the prosthesis 1300 than the lower portion of the struts 1326. The bottom portion of cells 1322 can be formed from a set of struts 1328 extending downwardly from a central or generally central location of each of the “V” shapes. The struts 1328 can extend along with a plane parallel to and/or extending through the longitudinal axis of the prosthesis 100. The geometry of cells 1322 can allow the cells 1322 to foreshorten as the outer frame 1304 is expanded, which can be used to secure the prosthesis to intralumenal tissue in or around the native valve.

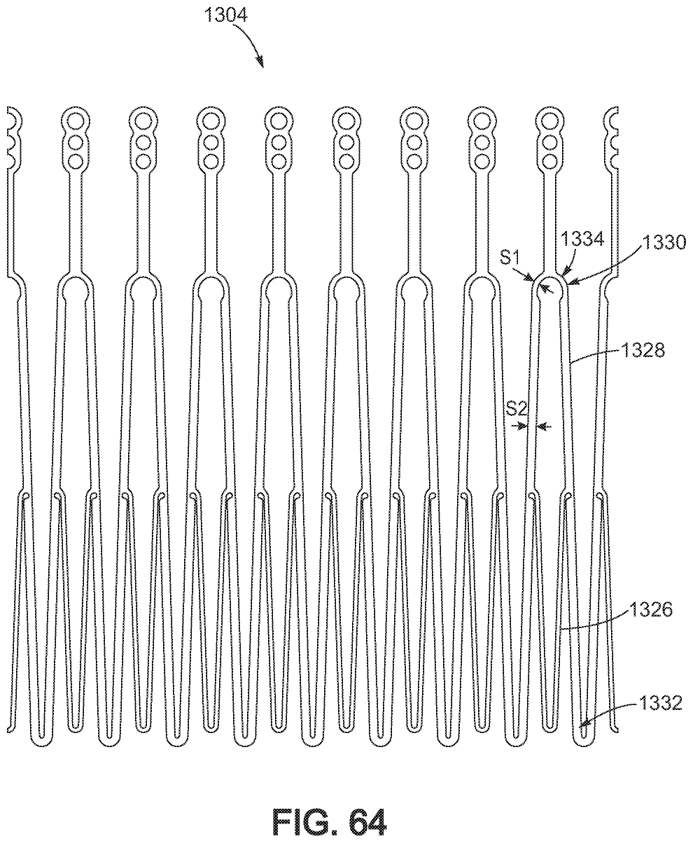

Any or all of the struts of the inner and/or outer frames of the prosthetic heart valve 1300 can comprise any of the varying strut width concepts described herein. For example, FIG. 64 illustrates the outer frame 1304 in a laid-flat configuration. In certain embodiments, the struts 1328 of the outer frame 1304 can comprise inflow end portions 1330 and outflow end portions 1332. The inflow end portions 1330 can define arcuate/round/circular apices or junctions 1334. At or proximate the junctions 1334, the struts 1328 can comprise a reduced strut width. For example, the inflow end portions 1330 of the struts can comprise a strut width S1, and the outflow end portions and intermediate portions between the inflow and outflow end portions can comprise a strut width S2 that is greater than the strut width S1. In certain embodiments, this can reduce the likelihood of infolding during recapture of the prosthetic valve 1300. In other embodiments, both the inflow and outflow end portions of the struts 1328 can comprise reduced strut widths as compared to the intermediate portions of the struts. The junctions 1334 can also comprise any of the radii and/or width dimensions described herein, and/or the width(s) of the struts 1328 can define any of the ratios described herein with the width and/or radii of the junctions 1334. In certain embodiments, the reduced strut widths shown in FIG. 64 can be implemented on the outflow end portions 1332 of the struts 1328. Reduced strut widths can also be implemented on any of the struts of the inner frame. Further details regarding the prosthetic heart valve 1300 can be found in U.S. Publication No. 2019/0262129, which is incorporated herein by reference.

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of the valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

### ADDITIONAL DESCRIPTION OF EXAMPLE EMBODIMENTS OF INTEREST

In view of the above described implementations of subject matter this application discloses the following list of examples, wherein one feature of an example in isolation or more than one feature of said example taken in combination and, optionally, in combination with one or more features of one or more further examples are further examples also falling within the disclosure of this application:

Example 1. A prosthetic implant, comprising a self-expanding frame having an inflow end, an outflow end, and a plurality of struts, the struts being interconnected at junctions, and wherein at least a portion of the plurality of struts have a reduced strut width at at least one junction.

Example 2. The prosthetic implant of any example herein and in particular example 1, wherein the struts of the at least a portion of the plurality of struts have a reduced strut width at both junctions.

Example 3. The prosthetic implant of any example herein and in particular example 1, wherein the struts of the at least a portion of the plurality of struts have a reduced strut width at their inflow junctions.

Example 4. The prosthetic implant of any example herein and in particular example 1, wherein the struts of the at least a portion of the plurality of struts have a reduced strut width at their outflow junctions.

Example 5. The prosthetic implant of any example herein and in particular any preceding example, wherein the struts define a first row of struts at the inflow end of the frame, a second row of struts at the outflow end of the frame, and at least one row of struts between the inflow end and the outflow end of the frame.

Example 6. The prosthetic implant of any example herein and in particular example 5, wherein struts of at least the first row of struts comprise a reduced strut width at their inflow junctions.

Example 7. The prosthetic implant of any example herein and in particular example 5 or example 6, wherein struts of at least the first row of struts comprise a reduced strut width at their outflow junctions.

Example 8. The prosthetic implant of any example herein and in particular any of examples 5-7, wherein struts of at least the second row of struts comprise a reduced strut width at their outflow junctions.

Example 9. The prosthetic implant of any example herein and in particular any of examples 5-8, wherein the struts of at least the second row of struts comprise a reduced strut width at their inflow junctions.

Example 10. The prosthetic implant of any example herein and in particular example 5, wherein the struts comprise inflow end portions, outflow end portions, and intermediate portions between the inflow end portions and the outflow end portions, and wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width.

Example 11. The prosthetic implant of any example herein and in particular example 10, wherein the third strut width is greater than the first strut width and greater than the second strut width.

Example 12. The prosthetic implant of any example herein and in particular example 10 or example 11, wherein the first strut width and the second strut width are substantially equal.

Example 13. The prosthetic implant of any example herein and in particular any of examples 10-12, wherein a ratio of the first strut width to the third strut width is less than or equal to 0.95, or from 0.7 to 0.95.

Example 14. The prosthetic implant of any example herein and in particular any of examples 10-13, wherein a ratio of the second strut width to the third strut width is less than or equal to 0.95, or from 0.7 to 0.95.

Example 15. The prosthetic implant of any example herein and in particular any of examples 10-14, wherein a thickness of the struts is greater than the third strut width.

Example 16. The prosthetic implant of any example herein and in particular example 15, wherein a ratio of the third strut width to the strut thickness is greater than or equal to 0.65, or from 0.65 to 0.85.

Example 17. The prosthetic implant of any example herein and in particular any of examples 10-16, wherein the junctions comprise a junction width, and the junction width is greater than the third strut width.

Example 18. The prosthetic implant of any example herein and in particular example 17, wherein a ratio of the third strut width to the junction width is 0.3 to 0.5.

Example 19. The prosthetic implant of any example herein and in particular example 17 or example 18, wherein the struts comprise a strut thickness, and the junction width is greater than the strut thickness.

Example 20. The prosthetic implant of any example herein and in particular example 19, wherein a ratio of the junction width to the strut thickness is less than or equal to 2.1, or from 1.5 to 2.1.

Example 21. The prosthetic implant of any example herein and in particular any preceding example, wherein when 80% of an overall length of the prosthetic implant is deployed from a delivery cylinder of a delivery apparatus, a ratio of a diameter of the inflow end of the prosthetic implant to an inner diameter of the delivery cylinder is less than or equal to 6.0, or 5.0 to 6.0.

Example 22. The prosthetic implant of any example herein and in particular any of examples 10-21, wherein the inflow end portions of the struts of the second row of struts comprise the first strut width, the outflow end portions of the struts of the second row of struts comprise the second strut width, and the intermediate portions of the struts of the second row of struts comprise the third strut width.

Example 23. The prosthetic implant of any example herein and in particular any of examples 10-22, wherein each junction comprises a curved inflow surface, the curved inflow surface defining a radius, and a ratio of the second strut width of the outflow ends of the struts to the radius of the curved inflow surface is 4.0 to 7.5.

Example 24. The prosthetic implant of any example herein and in particular any of examples 10-23, wherein all struts of the frame comprise the first strut width, the second strut width, and the third strut width.

Example 25. The prosthetic implant of any example herein and in particular any of examples 1-24, wherein the prosthetic implant is a prosthetic heart valve comprising a plurality of leaflets coupled to the frame and configured to regulate a flow of blood through the frame.

Example 26. The prosthetic implant of any example herein and in particular any of examples 1-24, wherein the prosthetic implant is a docking station configured to be implanted in an annulus of a native heart valve and configured to receive a prosthetic heart valve.

Example 27. A method, comprising advancing the prosthetic implant of any preceding claim from a delivery cylinder of a delivery apparatus in which the prosthetic implant is retained in a radially compressed state such that the inflow end of the prosthetic implant at least partially expands, and retracting the prosthetic implant back into the delivery cylinder such that the prosthetic implant returns to the radially compressed state.

Example 28. A prosthetic implant delivery apparatus, comprising a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant according to any example herein and in particular any of examples 1-26 retained in a radially compressed state in the delivery cylinder.

Example 29. The prosthetic implant delivery apparatus of any example herein and in particular example 28, wherein the prosthetic implant comprises a specified design diameter of at least 29 mm, and when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

Example 30. A prosthetic implant, comprising a self-expanding frame having an inflow end, an outflow end, and a plurality of struts, the struts being interconnected at junctions, wherein the struts define a first row of struts at the inflow end of the frame, a second row of struts at the outflow end of the frame, and at least one row of struts between the inflow end and the outflow end of the frame, wherein the struts comprise inflow end portions, outflow end portions, and intermediate portions between the inflow end portions and the outflow end portions, and wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width and greater than the second strut width.

Example 31. The prosthetic implant of any example herein and in particular example 30, wherein the first strut width and the second strut width are substantially equal.

Example 32. The prosthetic implant of any example herein and in particular example 30 or example 31, wherein a ratio of the first strut width to the third strut width is less than or equal to 0.95, or from 0.7 to 0.95.

Example 33. The prosthetic implant of any example herein and in particular any of examples 30-32, wherein a ratio of the second strut width to the third strut width is less than or equal to 0.95, or from 0.7 to 0.95.

Example 34. The prosthetic implant of any example herein and in particular any of examples 30-33, wherein a thickness of the struts is greater than the third strut width.

Example 35. The prosthetic implant of any example herein and in particular example 34, wherein a ratio of the third strut width to the strut thickness is greater than or equal to 0.65, or from 0.65 to 0.85.

Example 36. The prosthetic implant of any example herein and in particular any of examples 30-35, wherein the junctions comprise a junction width, and the junction width is greater than the third strut width.

Example 37. The prosthetic implant of any example herein and in particular example 36, wherein a ratio of the third strut width to the junction width is 0.3 to 0.5.

Example 38. The prosthetic implant of any example herein and in particular example 36 or example 37, wherein the struts comprise a strut thickness, and the junction width is greater than the strut thickness.

Example 39. The prosthetic implant of any example herein and in particular example 38, wherein a ratio of the junction width to the strut thickness is less than or equal to 2.1, or from 1.5 to 2.1.

Example 40. The prosthetic implant of any example herein and in particular any of examples 30-39, wherein when 80% of an overall length of the prosthetic implant is deployed from a delivery cylinder of a delivery apparatus, a ratio of a diameter of the inflow end of the prosthetic implant to an inner diameter of the delivery cylinder is less than or equal to 6.0, or 5.0 to 6.0.

Example 41. The prosthetic implant of any example herein and in particular any of examples 30-40, wherein the inflow end portions of the struts of the second row of struts comprise the first strut width, the outflow end portions of the struts of the second row of struts comprise the second strut width, and the intermediate portions of the struts of the second row of struts comprise the third strut width.

Example 42. The prosthetic implant of any example herein and in particular any of examples 30-41, wherein each junction comprises a curved inflow surface, the curved inflow surface defining a radius, and a ratio of the second strut width of the outflow ends of the struts to the radius of the curved inflow surface is 4.0 to 7.5.

Example 43. The prosthetic implant of any example herein and in particular any of examples 30-42, wherein all struts of the frame comprise the first strut width, the second strut width, and the third strut width.

Example 44. The prosthetic implant of any example herein and in particular any of examples 30-43, wherein the prosthetic implant is a prosthetic heart valve comprising a plurality of leaflets coupled to the frame and configured to regulate a flow of blood through the frame.

Example 45. The prosthetic implant of any example herein and in particular any of examples 30-43, wherein the prosthetic implant is a docking station configured to be implanted in an annulus of a native heart valve, and configured to receive a prosthetic heart valve.

Example 46. A method, comprising advancing the prosthetic implant of any example herein and in particular any of examples 30-45 from a delivery cylinder of a delivery apparatus in which the prosthetic implant is retained in a radially compressed state such that the inflow end of the prosthetic implant at least partially expands, and retracting the prosthetic implant back into the delivery cylinder such that the prosthetic implant returns to the radially compressed state.

Example 47. A prosthetic implant delivery apparatus, comprising a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant according to any example herein and in particular any of examples 30-45 retained in a radially compressed state in the delivery cylinder.

Example 48. The prosthetic implant delivery apparatus of any example herein and in particular example 47, wherein the prosthetic implant comprises a specified design diameter of at least 29 mm, and when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

Example 49. A prosthetic implant, comprising a self-expanding frame having an inflow end, an outflow end, and a plurality of struts, the struts being interconnected at junctions, wherein the struts comprise inflow end portions coupled to respective junctions, outflow end portions coupled to respective junctions, and intermediate portions between the inflow end portions and the outflow end portions, wherein a strut width of the intermediate portions of the struts is different from a strut width of the inflow end portions of the struts and different from a strut width of the outflow end portions of the struts, wherein the struts comprise a strut thickness, and wherein a ratio of the strut width of the intermediate portions of the struts to the strut thickness is greater than or equal to 0.65.

Example 50. The prosthetic implant of any example herein and in particular example 49, wherein the ratio of the strut width of the intermediate portions of the struts to the strut thickness is 0.65 to 0.85.

Example 51. The prosthetic implant of any example herein and in particular example 49 or example 50, wherein the struts define a first row of struts at the inflow end of the frame, a second row of struts at the outflow end of the frame, and at least one row of struts between the inflow end and the outflow end of the frame, and the inflow end portions of the struts of the first row of struts comprise a first strut width, the outflow end portions of the struts of the first row of struts comprise a second strut width, and the strut width of the intermediate portions of the struts of the first row of struts is a third strut width, the third strut width being greater than the first strut width and greater than the second strut width.

Example 52. The prosthetic implant of any example herein and in particular example 51, wherein all struts of the frame comprise the first strut width, the second strut width, and the third strut width.

Example 53. The prosthetic implant of any example herein and in particular example 51 or example 52, wherein the first strut width and the second strut width are substantially equal.

Example 54. The prosthetic implant of any example herein and in particular any of examples 51-53, wherein a ratio of the first strut width to the third strut width is less than or equal to 0.95, or from 0.7 to 0.95.

Example 55. The prosthetic implant of any example herein and in particular any of examples 51-54, wherein a ratio of the second strut width to the third strut width is less than or equal to 0.95, or from 0.7 to 0.95.

Example 56. The prosthetic implant of any example herein and in particular any of examples 51-55, wherein a thickness of the struts is greater than the third strut width.

Example 57. The prosthetic implant of any example herein and in particular any of examples 49-56, wherein the junctions comprise a junction width, and the junction width is greater than the strut width of the intermediate portions of the struts.

Example 58. The prosthetic implant of any example herein and in particular example 57, wherein a ratio of the strut width of the intermediate portions of the struts to the junction width is 0.3 to 0.5.

Example 59. The prosthetic implant of any example herein and in particular example 57 or example 58, wherein the struts comprise a strut thickness, and the junction width is greater than the strut thickness.

Example 60. The prosthetic implant of any example herein and in particular example 59, wherein a ratio of the junction width to the strut thickness is less than or equal to 2.1, or from 1.5 to 2.1.

Example 61. The prosthetic implant of any example herein and in particular any of examples 49-60, wherein when 80% of an overall length of the prosthetic implant is deployed from a delivery cylinder of a delivery apparatus, a ratio of a diameter of the inflow end of the prosthetic implant to an inner diameter of the delivery cylinder is less than 6.0, or from 5.0 to 6.0.

Example 62. The prosthetic implant of any example herein and in particular example 51, wherein the outflow end portions of the struts of the second row of struts comprise the first strut width, the outflow end portions of the struts of the second row of struts comprise the second strut width, and the intermediate portions of the struts of the second row of struts comprise the third strut width.

Example 63. The prosthetic implant of any example herein and in particular any of examples 49-62, wherein each junction comprises a curved inflow surface, the curved inflow surface defining a radius, and a ratio of the strut width of the outflow ends of the struts to the radius of the curved inflow surface is 4.0 to 7.5.

Example 64. The prosthetic implant of any example herein and in particular any of examples 49-63, wherein the prosthetic implant is a prosthetic heart valve comprising a plurality of leaflets coupled to the frame and configured to regulate a flow of blood through the frame.

Example 65. The prosthetic implant of any example herein and in particular any of examples 49-63, wherein the prosthetic implant is a docking station configured to be implanted in an annulus of a native heart valve, and configured to receive a prosthetic heart valve.

Example 66. A method, comprising advancing the prosthetic implant of any example herein and in particular any of examples 49-65 from a delivery cylinder of a delivery apparatus in which the prosthetic implant is retained in a radially compressed state such that the inflow end of the prosthetic implant at least partially expands, and retracting the prosthetic implant back into the delivery cylinder such that the prosthetic implant returns to the radially compressed state.

Example 67. A prosthetic implant delivery apparatus, comprising a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant according to any example herein and in particular any of examples 49-65 retained in a radially compressed state in the delivery cylinder.

Example 68. The prosthetic implant delivery apparatus of any example herein and in particular example 67, wherein the prosthetic implant comprises a specified design diameter of at least 29 mm, and when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

Example 69. A prosthetic implant, comprising a self-expanding frame having an inflow end, an outflow end, and a plurality of struts, the struts being interconnected at junctions, the junctions comprising a junction width, wherein the struts comprise inflow end portions coupled to respective junctions, outflow end portions coupled to respective junctions, and intermediate portions between the inflow end portions and the outflow end portions, wherein the inflow end portions of the struts comprise a first strut width, the outflow end portions of the struts comprise a second strut width, and the intermediate portions of the struts comprise a third strut width that is greater than the first strut width and greater than the second strut width, and wherein the junction width is greater than the third strut width of the intermediate portions of the struts.

Example 70. The prosthetic implant of any example herein and in particular example 69, wherein a ratio of the third strut width to the junction width is 0.3 to 0.5.

Example 71. The prosthetic implant of any example herein and in particular example 69 or example 70, wherein the first strut width and the second strut width are substantially equal.

Example 72. The prosthetic implant of any example herein and in particular any of examples 69-71, wherein a ratio of the first strut width to the third strut width is less than or equal to 0.95, or from 0.7 to 0.95.

Example 73. The prosthetic implant of any example herein and in particular any of examples 69-72, wherein a ratio of the second strut width to the third strut width is less than or equal to 0.95, or from 0.7 to 0.95.

Example 74. The prosthetic implant of any example herein and in particular any of examples 69-73, wherein a thickness of the struts is greater than the third strut width.

Example 75. The prosthetic implant of any example herein and in particular example 74, wherein a ratio of the third strut width to the strut thickness is greater than or equal to 0.65, or from 0.65 to 0.85.

Example 76. The prosthetic implant of any example herein and in particular example 74 or example 75, wherein the junction width is greater than the strut thickness.

Example 77. The prosthetic implant of any example herein and in particular example 76, wherein a ratio of the junction width to the strut thickness is less than or equal to 2.1, or from 1.5 to 2.1.

Example 78. The prosthetic implant of any example herein and in particular any of examples 69-77, wherein when 80% of an overall length of the prosthetic implant is deployed from a delivery cylinder of a delivery apparatus, a ratio of a diameter of the inflow end of the prosthetic implant to an inner diameter of the delivery cylinder is less than 6.0, or from 5.0 to 6.0.

Example 79. The prosthetic implant of any example herein and in particular any of examples 69-78, wherein the struts define a first row of struts at the inflow end of the frame, a second row of struts at the outflow end of the frame, and at least one row of struts between the inflow end and the outflow end of the frame, and the inflow end portions of the struts of the first row of struts comprise the first strut width, the outflow end portions of the struts of the first row of struts comprise the second strut width, and intermediate portions of the struts of the first row of struts comprise the third strut width, the third strut width being greater than the first strut width and greater than the second strut width.

Example 80. The prosthetic implant of any example herein and in particular example 79, wherein the inflow end portions of the struts of the second row of struts comprise the first strut width, the outflow end portions of the struts of the second row of struts comprise the second strut width, and the intermediate portions of the struts of the second row of struts comprise the third strut width.

Example 81. The prosthetic implant of any example herein and in particular any of examples 69-80, wherein each junction comprises a curved inflow surface, the curved inflow surface defining a radius, and a ratio of the second strut width of the outflow ends of the struts to the radius of the curved inflow surface is 4.0 to 7.5.

Example 82. The prosthetic implant of any example herein and in particular any of examples 69-81, wherein all struts of the frame comprise the first strut width, the second strut width, and the third strut width.

Example 83. The prosthetic implant of any example herein and in particular any of examples 69-82, wherein the prosthetic implant is a prosthetic heart valve comprising a plurality of leaflets coupled to the frame and configured to regulate a flow of blood through the frame.

Example 84. The prosthetic implant of any example herein and in particular any of examples 69-82, wherein the prosthetic implant is a docking station configured to be implanted in an annulus of a native heart valve, and configured to receive a prosthetic heart valve.

Example 85. A method, comprising, advancing the prosthetic implant of any example herein and in particular any of examples 69-84 from a delivery cylinder of a delivery apparatus in which the prosthetic implant is retained in a radially compressed state such that the inflow end of the prosthetic implant at least partially expands, and retracting the prosthetic implant back into the delivery cylinder such that the prosthetic implant returns to the radially compressed state.

Example 86. A prosthetic implant delivery apparatus, comprising a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant according to any example herein and in particular any of examples 69-84 retained in a radially compressed state in the delivery cylinder

Example 87. The prosthetic implant delivery apparatus of any example herein and in particular example 86, wherein the prosthetic implant comprises a specified design diameter of at least 29 mm, and when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

Example 88. A prosthetic implant delivery apparatus, comprising a catheter comprising a handle portion at a proximal end portion of the catheter and an elongated shaft extending from the handle portion, the catheter further comprising a delivery cylinder at a distal end portion of the shaft, the delivery cylinder comprising an inner diameter, and a self-expanding prosthetic implant retained in a radially compressed state in the delivery cylinder, the prosthetic implant comprising a self-expanding frame having an inflow end, an outflow end, and a plurality of struts, the struts being interconnected at junctions, and wherein the prosthetic implant has a specified design diameter of at least 29 mm, and wherein when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

Example 89. The prosthetic implant delivery apparatus of any example herein and in particular example 88, wherein the ratio of the diameter of the inflow end of the prosthetic implant to the inner diameter of the delivery cylinder is 5.0 to 6.0.

Example 90. The prosthetic implant delivery apparatus of any example herein and in particular example 88 or example 89, wherein at least a portion of the plurality of struts of the prosthetic implant have a reduced strut width at at least one junction.

Example 91. The prosthetic implant delivery apparatus of any example herein and in particular example 90, wherein the struts of the at least a portion of the plurality of struts have a reduced strut width at both junctions.

Example 92. The prosthetic implant delivery apparatus of any example herein and in particular example 90, wherein the struts of the at least a portion of the plurality of struts have a reduced strut width at their inflow junctions.

Example 93. The prosthetic implant of any example herein and in particular any of examples 90-92, wherein the struts of the at least a portion of the plurality of struts have a reduced strut width at their outflow junctions.

Example 94. The prosthetic implant delivery apparatus of any example herein and in particular any of examples 88-93, wherein the struts of the prosthetic implant define a first row of struts at the inflow end of the frame, a second row of struts at the outflow end of the frame, and at least one row of struts between the inflow end and the outflow end of the frame.

Example 95. The prosthetic implant delivery apparatus of any example herein and in particular example 94, wherein struts of at least the first row of struts comprise a reduced strut width at their inflow junctions.

Example 96. The prosthetic implant of any example herein and in particular example 94 or example 95, wherein struts of at least the first row of struts comprise a reduced strut width at their outflow junctions.

Example 97. The prosthetic implant of any example herein and in particular any of examples 94-96, wherein struts of at least the second row of struts comprise a reduced strut width at their outflow junctions.

Example 98. The prosthetic implant of any example herein and in particular example 97, wherein the struts of at least the second row of struts comprise a reduced strut width at their inflow junctions.

Example 99. The prosthetic implant delivery apparatus of any example herein and in particular example 94, wherein the struts comprise inflow end portions, outflow end portions, and intermediate portions between the inflow end portions and the outflow end portions, and wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width.

Example 100. The prosthetic implant delivery apparatus of any example herein and in particular example 99, wherein the third strut width is greater than the first strut width and greater than the second strut width.

Example 101. The prosthetic implant delivery apparatus of any example herein and in particular example 99 or example 100, wherein the first strut width and the second strut width are substantially equal.

Example 102. The prosthetic implant delivery apparatus of any example herein and in particular any of examples 99-106, wherein a ratio of the first strut width to the third strut width is less than or equal to 0.95, or from 0.7 to 0.95.

Example 103. The prosthetic implant delivery apparatus of any example herein and in particular any of examples 99-102, wherein a ratio of the second strut width to the third strut width is less than or equal to 0.95, or from 0.7 to 0.95.

Example 104. The prosthetic implant delivery apparatus of any example herein and in particular any of examples 99-103, wherein a thickness of the struts is greater than the third strut width.

Example 105. The prosthetic implant delivery apparatus of any example herein and in particular example 104, wherein a ratio of the third strut width to the strut thickness is greater than or equal to 0.65, or from 0.65 to 0.85.

Example 106. The prosthetic implant delivery apparatus of any example herein and in particular any of examples 99-105, wherein the junctions comprise a junction width, and the junction width is greater than the third strut width.

Example 107. The prosthetic implant delivery apparatus of any example herein and in particular example 106, wherein a ratio of the third strut width to the junction width is 0.3 to 0.5.

Example 108. The prosthetic implant delivery apparatus of any example herein and in particular example 106 or example 107, wherein the struts comprise a strut thickness, and the junction width is greater than the strut thickness.

Example 109. The prosthetic implant delivery apparatus of any example herein and in particular example 108, wherein a ratio of the junction width to the strut thickness is less than or equal to 2.1, or from 1.5 to 2.1.

Example 110. The prosthetic implant delivery apparatus of any example herein and in particular any of examples 99-109, wherein the inflow end portions of the struts of the second row of struts comprise the first strut width, the outflow end portions of the struts of the second row of struts comprise the second strut width, and the intermediate portions of the struts of the second row of struts comprise the third strut width.

Example 111. The prosthetic implant delivery apparatus of any example herein and in particular any of examples 99-110, wherein each junction comprises a curved inflow surface, the curved inflow surface defining a radius, and a ratio of the second strut width of the outflow ends of the struts to the radius of the curved inflow surface is 4.0 to 7.5.

Example 112. The prosthetic implant delivery apparatus of any example herein and in particular any of examples 99-111, wherein all struts of the frame comprise the first strut width, the second strut width, and the third strut width.

Example 113. The prosthetic implant delivery apparatus of any example herein and in particular any of examples 88-112, wherein the prosthetic implant is a prosthetic heart valve comprising a plurality of leaflets coupled to the frame and configured to regulate a flow of blood through the frame.

Example 114. The prosthetic implant delivery apparatus of any example herein and in particular any of examples 88-113, wherein the prosthetic implant is a docking station configured to be implanted in an annulus of a native heart valve, and configured to receive a prosthetic heart valve.

In view of the many possible embodiments to which the principles of the disclosed technology may be applied, it should be recognized that the illustrated embodiments are only preferred examples and should not be taken as limiting the scope of the disclosure. Rather, the scope of the disclosure is at least as broad as the following claims. We therefore claim all that comes within the scope and spirit of these claims.

1. A prosthetic implant comprising:
a frame having an inflow end, an outflow end, and a plurality of struts, the struts being interconnected at junctions, and wherein at least a portion of the plurality of struts have a reduced strut width at at least one junction.

2. The prosthetic implant of claim 1 wherein the frame is a self-expanding frame.

3. The prosthetic implant of claim 1, wherein the struts of the at least a portion of the plurality of struts have a reduced strut width at their inflow junctions.

4. The prosthetic implant of claim 1, wherein the struts of the at least a portion of the plurality of struts have a reduced strut width at their outflow junctions.

5. The prosthetic implant of claim 1, wherein the plurality of struts define a first row of struts at the inflow end of the frame, a second row of struts at the outflow end of the frame, and at least one row of struts between the inflow end and the outflow end of the frame.

6. The prosthetic implant of claim 5, wherein struts of at least the first row of struts comprise a reduced strut width at their inflow junctions.

7. The prosthetic implant of claim 5, wherein struts of at least the first row of struts comprise a reduced strut width at their outflow junctions.

8. The prosthetic implant of claim 5, wherein struts of at least the second row of struts comprise a reduced strut width at their inflow junctions.

9. The prosthetic implant of claim 5, wherein the struts comprise inflow end portions, outflow end portions, and intermediate portions between the inflow end portions and the outflow end portions; and
wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width.

10. The prosthetic implant of claim 9, wherein the third strut width is greater than the first strut width and greater than the second strut width.

11. The prosthetic implant of claim 9, wherein the first strut width and the second strut width are substantially equal.

12. The prosthetic implant of claim 9, wherein a ratio of the first strut width to the third strut width is less than or equal to 0.95, or from 0.7 to 0.95.

13. The prosthetic implant of claim 1, wherein when 80% of an overall length of the prosthetic implant is deployed from a delivery cylinder of a delivery apparatus, a ratio of a diameter of the inflow end of the prosthetic implant to an inner diameter of the delivery cylinder is less than or equal to 6.0, or 5.0 to 6.0.

14. The prosthetic implant of claim 1, wherein the prosthetic implant is a prosthetic heart valve comprising a plurality of leaflets coupled to the frame and configured to regulate a flow of blood through the frame.

15. The prosthetic implant of claim 1, wherein the prosthetic implant is a docking station configured to be implanted in an annulus of a native heart valve and configured to receive a prosthetic heart valve.

16. The prosthetic implant of claim 1 wherein the prosthetic implant is a balloon-expandable prosthetic valve adapted to be mounted in a compressed state on the balloon of a delivery catheter.

17. A prosthetic implant delivery apparatus, comprising:
a catheter comprising a handle portion at a proximal end portion of the catheter and an elongated shaft extending from the handle portion, the catheter further comprising a delivery cylinder at a distal end portion of the shaft, the delivery cylinder comprising an inner diameter; and
a prosthetic implant comprising a self-expanding frame having an inflow end, an outflow end, and a plurality of struts, the struts being interconnected at junctions, and wherein at least a portion of the plurality of struts have a reduced strut width at at least one junction, the prosthetic implant retained in a radially compressed state in the delivery cylinder.

18. The prosthetic implant delivery apparatus of claim 17, wherein:
the prosthetic implant comprises a specified design diameter of at least 29 mm; and
when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

19. A prosthetic implant, comprising:
a self-expanding frame having an inflow end, an outflow end, and a plurality of struts, the struts being interconnected at junctions,
wherein the struts define a first row of struts at the inflow end of the frame, a second row of struts at the outflow end of the frame, and at least one row of struts between the inflow end and the outflow end of the frame;
wherein the struts comprise inflow end portions, outflow end portions, and intermediate portions between the inflow end portions and the outflow end portions; and
wherein the inflow end portions of the struts of the first row of struts comprise a first strut width, the outflow end portions of the struts of the first row of struts comprise a second strut width, and the intermediate portions of the struts of the first row of struts comprise a third strut width that is greater than the first strut width and greater than the second strut width.

20. The prosthetic implant of claim 19, wherein the first strut width and the second strut width are substantially equal.

21. The prosthetic implant of claim 19, wherein a ratio of the first strut width to the third strut width is less than or equal to 0.95, or from 0.7 to 0.95.

22. The prosthetic implant of claim 19, wherein the prosthetic implant is a prosthetic heart valve comprising a plurality of leaflets coupled to the frame and configured to regulate a flow of blood through the frame or a docking station configured to be implanted in an annulus of a native heart valve, and configured to receive a prosthetic heart valve.

23. A prosthetic implant delivery apparatus, comprising:
a catheter comprising a handle portion at a proximal end portion of the catheter and an elongated shaft extending from the handle portion, the catheter further comprising a delivery cylinder at a distal end portion of the shaft, the delivery cylinder comprising an inner diameter; and
a self-expanding prosthetic implant retained in a radially compressed state in the delivery cylinder, the prosthetic implant comprising:
a self-expanding frame having an inflow end, an outflow end, and a plurality of struts, the struts being interconnected at junctions; and
wherein the prosthetic implant has a specified design diameter of at least 29 mm; and
wherein when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.

24. A method, comprising:
advancing a self-expanding prosthetic implant from a delivery cylinder of a delivery apparatus in which the prosthetic implant is retained in a radially compressed state such that the inflow end of the prosthetic implant at least partially expands; and
retracting the prosthetic implant back into the delivery cylinder such that the prosthetic implant returns to the radially compressed state;
wherein when the prosthetic implant is partially deployed from the delivery cylinder such that at least 80% of an overall length of the prosthetic implant is unsheathed, a ratio of a diameter of the inflow end of the prosthetic implant to the inner diameter of the delivery cylinder is less than or equal to 6.0.
